(19) United States
(12) Patent Application Publication
(10) Pub. No.: US 2013/0071439 A1
(43) Pub. Date: Mar. 21, 2013

(54) METHODS AND COMPOSITIONS FOR TREATING BIOFILMS

(71) Applicants: Richard Losick, Lexington, MA (US); Jon Clardy, Jamaica Plain, MA (US); Roberto Kolter, Cambridge, MA (US); Illana Kolodkin-Gal, Cambridge, MA (US); Diego Romero, Boston, MA (US); Shugeng Cao, Waltham, MA (US)

(72) Inventors: Richard Losick, Lexington, MA (US); Jon Clardy, Jamaica Plain, MA (US); Roberto Kolter, Cambridge, MA (US); Illana Kolodkin-Gal, Cambridge, MA (US); Diego Romero, Boston, MA (US); Shugeng Cao, Waltham, MA (US)

(73) Assignee: PRESIDENT AND FELLOWS OF HARVARD COLLEGE, Cambridge, MA (US)

(21) Appl. No.: 13/520,745

(22) PCT Filed: Jan. 10, 2011
(86) PCT No.: PCT/US11/20705

§ 371 Date: Nov. 21, 2012

Related U.S. Application Data
(60) Provisional application No. 61/293,414, filed on Jan. 8, 2010.
(60) Provisional application No. 61/329,930, filed on Apr. 30, 2010.

(51) Int. Cl. A61K 31/198; A61K 33/00; A61K 8/44; A61K 31/4172; A61Q 11/00; C07C 229/08; C07C 323/58; C07C 229/24; C07D 233/64; C07C 237/06; C07C 229/26; C07C 229/22; C07D 209/20; C07C 229/34; C07D 207/16; C07C 279/14; A61K 9/00; A61P 31/04; A61P 11/00; A61P 17/00; A61P 13/02; A61P 15/02; A61K 31/405

(52) U.S. Cl. 424/400; 514/561; 514/419; 424/600; 424/54; 514/563; 514/400; 514/564; 514/567; 514/565; 562/557; 562/571; 548/341.5; 562/561; 562/575; 562/563; 562/562; 562/570; 548/496; 562/444; 548/535; 562/560

(57) ABSTRACT

Methods of treating or reducing biofilms, treating a biofilm-related disorder, and preventing biofilm formation using D-amino acids are described.

### PRIORITY

This application claims priority to co-pending U.S. Provisional Application No. 61/293,414, filed Jan. 8, 2010, and U.S. Provisional Application No. 61/329,930, filed Apr. 30, 2010.

The application is related to copending International Patent Application filed on even date herewith and entitled “Method and CoatingComposition for Treating Biofilms.”

The contents of those applications are incorporated by reference.

### STATEMENT OF GOVERNMENT RIGHTS

This invention was made with United States Government support under the National Institutes of Health awards CA24487, GM058213, GM082137, GM086258, and GM18568. The United States government has certain rights in the invention.

### BACKGROUND

Biofilms are communities of cells that settle and proliferate on surfaces and are covered by an exopolymer matrix. They are slow-growing and many are in the stationary phase of growth. They can be formed by most, if not all, pathogens. According to the CDC, 65% of all infections in the United States are caused by biofilms that can be formed by common pathogens. Biofilms are also found in industrial settings, such as in drinking water distribution systems.

### SUMMARY

Aspects of the invention feature methods of treating, reducing, or inhibiting biofilm formation by bacteria. In some embodiments, the method comprises contacting a surface with a composition comprising an effective amount of a D-amino acid, thereby treating, reducing or inhibiting formation of the biofilm. In some embodiments, the bacteria are Gram-negative or Gram-positive bacteria. In particular embodiments, the bacteria are Bacillus, Staphylococcus, E. coli, or Pseudomonas bacteria.

In other aspects, the invention features compositions, such as industrial, therapeutic or pharmaceutical compositions, comprising one or more D-amino acids. In certain embodiments, the composition comprises D-tyrosine, D-leucine, D-methionine, D-tryptophan, or a combination thereof. In some embodiments, the composition comprises D-tyrosine, D-phenylalanine, D-proline, or a combination thereof. In further embodiments, the composition comprises two or more of D-tyrosine, D-leucine, D-phenylalanine, D-methionine, D-proline, and D-tryptophan, and in yet further embodiments the latter composition is essentially free of detergent and/or L amino acids. In other embodiments, the composition is used to treat an industrial biofilm described herein, such as in water treatment or plumbing systems.

In some embodiments, the composition is essentially free of L-amino acids. For example, the composition comprises less than 30%, less than 20%, less than 10%, less than 5%, less than 1%, less than 0.5%, less than 0.25%, less than 0.1%, less than 0.05%, less than 0.025%, less than 0.01%, less than 0.005%, less than 0.0025%, less than 0.001%, or less, of L-amino acids.

In some embodiments, the composition is essentially free of detergent. For example, the composition comprises less than 30%, less than 20%, less than 10%, less than 5%, less than 1%, less than 0.5%, less than 0.25%, less than 0.1%, less than 0.05%, less than 0.025%, less than 0.01%, less than 0.005%, less than 0.0025%, less than 0.001%, or less, of a detergent.

Another aspect of this disclosure is directed to methods of treating a biofilm-related disorder in a subject in need thereof, the method comprising administering to the subject a composition comprising an effective amount of a D-amino acid or a combination of D-amino acids, thereby treating the biofilm-related disorder, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or wherein the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-tyrosine.utamic acid, D-phenylalanine, D-histidine, D-isoleucine, D-lysine, D-leucine, D-asparagine, D-proline, D-glutamine, D-arginine, D-serine, D-threonine, D-valine, D-tryptophan, and D-tyrosine. In some embodiments, the composition is administered to a surface of the subject selected from the group of dermal and mucosal surfaces and combinations thereof. In other embodiments, the surface is an oral surface, a skin surface, a urinary tract surface, a vaginal tract surface, or a lung surface.

In some embodiments, the composition is essentially free of the corresponding L-amino acid or L-amino acids relative to the D-amino acids or combination of D-amino acids.

In some embodiments, the composition is administered to the subject via subcutaneous, intra-muscular, intra-peritoneal, intravenous, oral, nasal, or topical administration, and a combination thereof.

In some embodiments, the subject is a human.

In some embodiments, the formation of a biofilm is inhibited. In other embodiments, a previously formed biofilm is disrupted.

In some embodiments, the D-amino acid is administered at a concentration of about 0.1 nM to about 100 μM, for example, at a concentration of 0.1 nM to 100 μM.

In further embodiments, the biofilm-related disorder is selected from the group consisting of pneumonia, cystic fibrosis, otitis media, chronic obstructive pulmonary disease, and a urinary tract infection and combinations thereof. In other embodiments, the biofilm-related disorder is a medical device-related infection. In further embodiments, the biofilm-related disorder is a periodontal disease, such as gingivitis, periodontitis or breath malodor. In still further embodiments, the biofilm-related disorder is caused by bacteria. In some embodiments, the bacteria are Gram-negative or Gram-positive bacteria. In still other embodiments, the bacteria are of the genus Actinobacillus, Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Helicobacter, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia, or Xanthomonas.

Another aspect of this disclosure is directed to methods of treating, reducing, or inhibiting biofilm formation by biofilm forming bacteria on a biologically-related surface, the method comprising contacting a biological surface with a composition comprising an effective amount of a D-amino acid or a combination of D-amino acids, thereby treating, reducing or inhibiting formation of the biofilm, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or wherein the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the composition is essentially free of the corresponding L-amino acid or L-amino acids relative to the D-amino acids or combination of D-amino acids.

In some embodiments, the bacteria are Gram-negative or Gram-positive bacteria. In some embodiments, the bacteria are of the genus Actinobacillus, Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Helicobacter, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia, or Xanthomonas.

In some embodiments, the surface comprises a medical device, a wound dressing, a contact lens, or an oral device. In other embodiments, the medical device is selected from the group consisting of a clamp, forcep, scissors, skin hook, tubing, needle, retractor, scaler, drill, chisel, rasp, saw, catheter, orthopedic device, artificial heart valve, prosthetic joint, voice prosthetic, stent, shunt, pacemaker, surgical pin, respirator, ventilator, and an endoscope and combinations thereof.

In some embodiments of the foregoing methods, the composition comprises D tyrosine. In addition to D-tyrosine, in some embodiments, the composition further comprises one or more of D proline and D phenylalanine. In still other embodiments, in addition to D-tyrosine, the composition further comprises one or more of D-leucine, D-tryptophan, and D-methionine. In still further embodiments, in addition to D-tyrosine, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-tyrosine.utamic acid, D-phenylalanine, D-histidine, D-isoleucine, D-lysine, D-leucine, D-asparagine, D-proline, D-glutamine, D-arginine, D-serine, D-threonine, D-valine, and D-tryptophan.

In some embodiments of any of the foregoing methods, the method further comprises administering a biocide. In some embodiments, the biocide is an antibiotic.

In still other embodiments, the composition is essentially free of detergent.

Yet another aspect of the invention is directed to compositions comprising a D-amino acid or a mixture of D-amino acids in an amount effective to treat, reduce, or inhibit biofilm formation, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof or wherein the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-tyrosine.utamic acid, D-phenylalanine, D-histidine, D-isoleucine, D-lysine, D-leucine, D-asparagine, D-proline, D-glutamine, D-arginine, D-serine, D-threonine, D-valine, D-tryptophan, D-tyrosine.

In some embodiments, the composition is essentially free of the corresponding L-amino acid or L-amino acids relative to the D-amino acids or combination of D-amino acids.

In some embodiments, the D-amino acid is D tyrosine. In other embodiments the composition further comprises one or more of D proline and D phenylalanine. In still other embodiments, the composition further comprises one or more of D-leucine, D-tryptophan, and D-methionine. In further embodiments, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-tyrosine.utamic acid, D-phenylalanine, D-histidine, D-isoleucine, D-lysine, D-leucine, D-asparagine, D-proline, D-glutamine, D-arginine, D-serine, D-threonine, D-valine, and D-tryptophan.

In some embodiments, any of the foregoing compositions can also comprise polyhexamethylene biguanide, chlorhexidine, xylitol, triclosan, or chlorine dioxide. In other embodiments, any of the foregoing compositions can also comprise a pharmaceutically acceptable carrier. In still other embodiments of any the foregoing compositions, the effective amount is an amount effective to treat or prevent a biofilm-related disorder. In some embodiments, an effective amount comprises and amount effective to treat or prevent a biofilm on a surface.

In yet other embodiments of any the foregoing compositions, the biofilm-related disorder is pneumonia, cystic fibrosis, otitis media, chronic obstructive pulmonary disease, or a urinary tract infection. In some embodiments, the biofilm-related disorder is a medical device-related infection.

In some embodiments of any of the foregoing compositions, the composition further comprises an agent suitable for application to the surface. In other embodiments of any of the foregoing compositions, the composition is formulated as a wash solution, a dressing, a wound gel, or a synthetic tissue. In further embodiments, the composition is formulated as tablets, pills, troches, capsules, aerosol spray, solutions, suspensions, gels, pastes, creams, or foams. In some embodiments, the composition is formulated for parenteral, e.g., intravenous, intradermal, subcutaneous, oral (e.g., inhalation), transdermal (topical), transmucosal, vaginal and rectal administration.

Another aspect of this disclosure is directed to biofilm resistant medical devices, comprising a surface likely to contact a biological fluid, and a D-amino acid or a combination of D-amino acids coated on or impregnated into said surface, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or wherein the combination of D-amino acids is in an amount effective to treat, reduce, or inhibit biofilm formation, wherein the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-tyrosine.utamic acid, D-phenylalanine, D-histidine, D-isoleucine, D-lysine, D-leucine, D-asparagine, D-proline, D-glutamine, D-arginine, D-serine, D-threonine, D-valine, D-tryptophan, D-tyrosine.

In some embodiments, the D-amino acid is D-tyrosine or the combination of D-amino acids comprises D tyrosine. In other embodiments, the composition further comprises one or more of D proline and D phenylalanine. In other embodiments, the composition further comprises one or more of D-leucine, D-tryptophan, and D-methionine. In some embodiments, the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-tyrosine.utamic acid, D-phenylalanine, D-histidine, D-isoleucine, D-lysine, D-leucine, D-asparagine, D-proline, D-glutamine, D-arginine, D-serine, D-threonine, D-valine, and D-tryptophan.

In some embodiments, the D-amino acid is formulated as a slow-release formulation. In some embodiments, the surface is essentially free of L-amino acids. In further embodiments, the surface is essentially free of detergent.

In some embodiments, the device is selected from one or more of clamp, forcep, scissors, skin hook, tubing, needle, retractor, scaler, drill, chisel, rasp, saw, catheter, orthopedic device, artificial heart valve, prosthetic joint, voice prosthetic, stent, shunt, pacemaker, surgical pin, respirator, ventilator and endoscope.

A further aspect of the instant disclosure is directed to potable liquids comprising a D-amino acid or a combination of D-amino acids at a concentration in the range of 0.000001% to 0.1%, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or wherein the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-tyrosine.utamic acid, D-phenylalanine, D-histidine, D-isoleucine, D-lysine, D-leucine, D-asparagine, D-proline, D-glutamine, D-arginine, D-serine, D-threonine, D-valine, D-tryptophan, D-tyrosine.

Another aspect of this disclosure is directed to compositions resistant to biofilm formation, comprising a pharmaceutically or cosmetically suitable base, and an effective amount of a D-amino acid or a combination of D-amino acids distributed in the base, thereby treating, reducing or inhibiting formation of the biofilm, wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, and a combination thereof, or wherein the combination of D-amino acids is a synergistic combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

In some embodiments, the base is essentially free of the corresponding L-amino acid or L-amino acids relative to the D-amino acids or combination of D-amino acids.

In some embodiments, the base is selected from a liquid, gel, paste, or powder. In further embodiments, the composition is selected from the group consisting of shampoos, bath additives, hair care preparations, soaps, lotions, creams, deodorants, skin-care preparations, cosmetic personal care preparations, intimate hygiene preparations, foot care preparations, light protective preparations, skin tanning preparations, insect repellants, antiperspirants, sharing preparations, hair removal preparations, fragrance preparations, dental care, denture care and mouth care preparations and combinations thereof.

### BRIEF DESCRIPTION OF THE FIGURES

The following figures are presented for the purpose of illustration only, and are not intended to be limiting.

FIGS. 1A and 1B show cells of B. subtilis strain NCIB3610 that were grown at 22° C. in 12-well plates in liquid biofilm-inducing medium for 3 days (A) or for 8 days (B).

FIGS. 1C and 1D show cells grown for 3 days in medium to which had been added a dried and resuspended methanol eluate (1:100 v/v) from a C18 Sep Pak column that had been loaded with conditioned medium from a 6-8 day-old culture (C) or a 3 day-old culture (D). The final concentration of concentrated factor added to the wells represented a 1:4 dilution on a volume basis of the original conditioned media.

FIG. 1E is the same as FIG. 1C except the factor was further purified on the C-18 column by step-wise elution with methanol. Shown is the result of adding 3 μl of the 40% methanol eluate.

FIG. 1F is the same as FIG. 1C except that prior to addition to fresh medium the 40% methanol eluate was incubated with Proteinase K beads for 2 hours followed by centrifugation to remove the beads.

FIG. 2A shows the effects on pellicle formation of adding D-tyrosine (3 μM), D-leucine (8.5 mM), L-tyrosine (7 mM), or L-leucine (8.5 mM) to freshly inoculated cultures in biofilm-inducing medium after incubation for 3 days.

FIG. 2B shows the Minimal Biofilm Inhibitory Concentration (MBIC) of D-amino acids required for complete inhibition of pellicle formation.

FIG. 2C shows 3 day-old cultures to which had been added no amino acids (untreated), D-tyrosine (3 μM) or a mixture of D-tyrosine, D-tryptophan, D-methionine and D-leucine (2.5 nM each), followed by further incubation for 8 hours.

FIG. 2D shows the effect of concentrated Sep Pak C-18 column eluate from conditioned medium from an 8-day-old culture from the wild type or from a strain (IKG55) doubly mutant for ylmE and racX.

FIG. 2E shows S. aureus (strain SCO1) that had been grown in 12-well polystyrene plates for 24 hours at 37° C. in TSB medium containing glucose (0.5%) and NaCl (3%). Additionally added to the wells were no amino acids (untreated), D-tyrosine (50 μM) or the D-amino acid mixture (15 nM each). Cells bound to the polystyrene were visualized by washing away unbound cells and then staining with crystal violet.

FIG. 3A shows incorporation of radioactive D-tyrosine into the cell wall. Cells were grown in biofilm-inducing medium and incubated with either ¹⁴C-D-tyrosine or ¹⁴C-L-proline (10 μCi/ml) for 2 h at 37° C. Results are presented as a percent of total incorporation into cells (360,000 cpm/ml for L-proline and 46,000 cpm/ml for D-tyrosine).

FIG. 3B shows total fluorescence from cells (DR-30 (Romero et al., Proc. Natl. Acad. Sci. USA (2010, in press)) containing a functional tasA-mCherry translational fusion. The cells were grown to stationary phase with shaking in biofilm-inducing medium in the presence or absence of D-tyrosine (6 μM).

FIG. 3C shows cell association of TasA-mCherry by fluorescence microscopy. Wild-type cells and yqxM6 (IKG51) mutant cells containing the tasA-mCherry fusion were grown to stationary phase (OD=1.5) with shaking in biofilm-inducing medium in the presence or absence (untreated) of D-tyrosine (6 μM) as indicated, washed in PBS, and visualized by fluorescence microscopy.

FIG. 3D shows cell association of TasA fibers by electron microscopy. 24-hour-old cultures were incubated without (images 1 and 2) or with (images 3-6) D-tyrosine (0.1 mM) for an additional 12 hours. TasA fibers were stained by immunogold labeling using anti-TasA antibodies, and visualized by transmission electron microscopy as described in the Examples. The cells were mutant for the eps operon (Δeps) as the absence of exopolysaccharide significantly improves the imaging of TasA fibers. Filled arrows indicate fiber bundles; open arrows indicate individual fibers. The scale bar is 500 nm. The scale bar in the enlargements of images 2, 4 and 6 is 100 nm. Images 1 and 2 show fiber bundles attached to cells, images 3, 4 and 6 show individual fibers and bundles detached from cells, and images 3-5 show cells with little or no fiber material.

FIG. 4A shows cells grown for 3 days on solid (top images) or liquid (bottom images) biofilm-inducing medium that did or did not contain D-tyrosine.

FIG. 4B shows an abbreviated amino acid sequence for YqxM. Underlined are residues specified by codons in which the yqxM2 and yqxM6 frame-shift mutations resulted in the indicated sequence changes.

FIG. 5 shows wells containing MSgg medium supplemented with D-tryptophan (0.5 mM), D-methionine (2 mM), L-tryptophan (5 mM) or L-methionine (5 mM) that were inoculated with strain NCIB3610 and incubated for 3 days.

FIG. 6 shows plates containing solid MSgg medium supplemented with D-tyrosine (3 μM) or D-leucine (8.5 mM) that were inoculated with strain NCIB3610 and incubated for 4 days.

FIG. 7 shows NCIB3610 (WT) and a mutant doubly deleted for ylmE and racX (IKG155) that were grown in 12 well plates and incubated for 5 days.

FIG. 8 shows the effect of D-amino acids on cell growth. Cells were grown in MSgg medium containing D-tyrosine (3 μM), D-leucine (8.5 mM) or the four D-amino acids mixture (2.5 nM each) with shaking.

FIG. 9A shows the expression of P_{yqxM}-lacZ by strain FC122 (carrying P_{yqxM}-lacZ) and FIG. 9B shows the expression of P_{epsA}-lacZ by strain FC5 (carrying P_{epsA}-lacZ) that were grown in MSgg medium containing D-tyrosine (3 μM), D-leucine (8.5 mM) or the four D-amino acids mixture (2.5 nM each) with shaking.

FIG. 10 shows the inhibition of Pseudomonas aeruginosa biofilm formation by D-amino acids. P. aeruginosa strain P014 was grown in 12-well polystyrene plates for 48 hours at 30° C. in M63 medium containing glycerol (0.2%) and Casamino acids (20 μg/ml). Additionally added to the wells were no amino acids (untreated), D-tyrosine or the D-amino acid mixture. Cells bound to the polystyrene were visualized by washing away unbound cells and then staining with crystal violet. Wells were stained with 500 μl of 1.0% Crystal-violet dye, rinsed twice with 2 ml double-distilled water and thoroughly dried.

FIG. 11 shows crystal violet staining of Staphylococcus aureus biofilms grown with either individual D-amino acids or the quartet mixture in TSB medium for 24 hrs.

FIG. 12 shows crystal violet staining of Pseudomonas aeruginosa grown with either individual D-amino acids or the quartet mixture in M63 medium for 48 hrs.

FIG. 13 shows crystal violet staining of Staphylococcus aureus biofilms grown with either individual D-amino acids or a mixture in TSB medium for 24 hrs.

FIG. 14 shows crystal violet staining of Staphylococcus aureus biofilms grown in TSB medium with L-amino acids for 24 hrs.

FIG. 15 is a representative image of the Staphylococcus aureus biofilms formed in TSB medium applied with D-amino acids after removing planktonic bacteria.

FIG. 16 is a representative image of the Staphylococcus aureus biofilms formed in TSB medium applied with L-amino acids after removing planktonic bacteria.

FIG. 17 is a quantification of the cells within the Staphylococcus aureus biofilms formed in TSB medium after removing planktonic bacteria. Cells were re-suspended in PBS.

FIG. 18 shows the effect of D-aa mixture (1 mM) on Staphylococcus aureus biofilm formation on surfaces. Epoxy surfaces were soaked in D/L aa mixture and then incubated with bacteria for 24 hrs.

FIG. 19 shows the effect of D-aa mixture (1 mM) on Staphylococcus aureus biofilm formation on surfaces. Epoxy surfaces were soaked in D/L aa mixture and then incubated with bacteria for 24 hrs.

FIG. 20 shows the effect of D-aa on biofilm formation on M63 solid medium in Pseudomonas aeruginosa. Colonies were grown on room temperature for 4 days.

FIG. 21 shows the Sytox-staining of single attached cells in the button of 6 well plate of Pseudomonas aeruginosa in biofilm inducing conditions.

FIG. 22 shows crystal violet staining of Proteus mirabilis grown with either D-amino acids (100 μM) or the L-amino acids (100 μM) mixture in LB medium for 48 hrs.

FIG. 23 shows crystal violet staining of Streptococcus mutans grown either with D- or L-amino acids (1 mM) in BHI medium applied with sucrose (0.5%) medium for 72 hrs.

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims. As will be apparent to one of skill in the art, specific features and embodiments described herein can be combined with any other feature or embodiment.

### DEFINITIONS

The terms “disorder”, “disease”, and “condition” are used herein interchangeably for a condition in a subject. A disorder is a disturbance or derangement that affects the normal function of the body of a subject. A disease is a pathological condition of an organ, a body part, or a system resulting from various causes, such as infection, genetic defect, or environmental stress that is characterized by an identifiable group of symptoms. A disorder or disease can refer to a biofilm-related disorder that is characterized by a disease-related growth of bacteria in that a biofilm is established.

The terms “prevent,” “preventing,” and “prevention” refer herein to the inhibition of the development or onset of a biofilm or of a biofilm-related disorder or the prevention of the recurrence, onset, or development of one or more indications or symptoms of a biofilm or of a biofilm-related disorder on a surface or in a subject resulting from the administration of a composition described herein (e.g., a prophylactic or therapeutic composition), or the administration of a combination of therapies (e.g., a combination of prophylactic or therapeutic compositions).

As used herein, “treat”, “treating” or “treatment” refers to administering a composition described herein in an amount, manner (e.g., schedule of administration), and/or mode (e.g., route of administration), effective to improve a disorder or a symptom thereof, or to prevent or slow the progression of a disorder or a symptom thereof. This can be evidenced by, e.g., an improvement in a parameter associated with a biofilm or with a biofilm-related disorder or an indication or symptom thereof, e.g., to a statistically significant degree or to a degree detectable to one skilled in the art. An effective amount, manner, or mode can vary depending on the surface, application, and/or subject and may be tailored to the surface, application, and/or subject. By preventing or slowing progression of a biofilm or of a biofilm-related disorder or an indication or symptom thereof, a treatment can prevent or slow deterioration resulting from a biofilm or from a biofilm-related disorder or an indication or symptom thereof on an effected surface or in an affected or diagnosed subject.

The invention is based, at least in part, on the discovery that D-amino acids present in conditioned medium from mature biofilms prevents biofilm formation and triggers the disassembly of existing biofilms. Standard amino acids can exist in either of two optical isomers, called L- or D-amino acids, which are mirror images of each other. While L-amino acids represent the vast majority of amino acids found in proteins, D-amino acids are components of the peptidoglycan cell walls of bacteria.

The D-amino acids described herein are capable of penetrating biofilms on living and non-living surfaces, of preventing the adhesion of bacteria to surfaces and any further build-up of the biofilm, of detaching such biofilm and/or inhibiting the further growth of the biofilm-forming micro-organisms in the biological matrix, or of killing such micro-organisms. D-amino acids are known in the art and can be prepared using known techniques. Exemplary methods include, e.g., those described in U.S. Publ. No. 20090203091. D-amino acids are also commercially available (e.g., from Sigma Chemicals, St. Louis, Mo.).

Any D-amino acid can be used in the methods described herein, including without limitation D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, or D-tyrosine. A D-amino acid can be used alone or in combination with other D-amino acids. In exemplary methods, 2, 3, 4, 5, 6, or more D-amino acids are used in combination. Preferably, D-tyrosine, D-leucine, D-methionine, or D-tryptophan, either alone or in combination, are used in the methods described herein. In other preferred embodiments, D-tyrosine, D-proline and D-phenylalanine, either alone or in combination, are used in the methods described herein.

A D-amino acid can be administered at a concentration of 0.1 nM to 100 μM, e.g., 1 nM to 10 μM, 5 nM to 5 μM, or 10 nM to 1 μM. In other embodiments, a D-amino acid can be administered at a concentration of about 0.1 nM to about 100 μM, e.g., about 1 nM to about 10 μM, about 5 nM to about 5 μM, or about 10 nM to about 1 μM.

An exemplary D-amino acid composition found to be particularly effective in inhibiting or treating biofilm formation includes D-tyrosine. In some embodiments, D-tyrosine is used alone and can be used, for example, as concentrations of less than 1 mM, or less than 100 μM or less than 10 μM, or at a concentration of 0.1 nM to 100 μM, e.g., 1 nM to 10 μM, 5 nM to 5 μM, or 10 nM to 1 μM.

In other embodiments, D-tyrosine is used in combination with one or more of D-proline and D-phenylalanine. In some embodiments, D-tyrosine is used in combination with one or more of D-leucine, D-tryptophan, and D-methionine. The combinations of D-tyrosine with one or more of D-proline, D-phenylalanine, D-leucine, D-tryptophan, and D-methionine can be synergistic and can be effective in inhibiting or treating biofilm formation at total D-amino acid concentrations of 10 μM or less, e.g., about 1 nM to about 10 μM, about 5 nM to about 5 μM, or about 10 nM to about 1 μM, or at a concentration of 0.1 nM to 100 μM, e.g., 1 nM to 10 μM, 5 nM to 5 μM, or 10 nM to 1 μM.

In some embodiments, the combinations of D-amino acids are equimolar. In other embodiments, the combinations of D-amino acids are not in equimolar amounts.

In some embodiments, the composition is essentially free of L-amino acids. For example, the composition comprises less than about 30%, less than about 20%, less than about 10%, less than about 5%, less than about 1%, less than about 0.5%, less than about 0.25%, less than about 0.1%, less than about 0.05%, less than about 0.025%, less than about 0.01%, less than about 0.005%, less than about 0.0025%, less than about 0.001%, or less, of L-amino acids. In other embodiments, the composition comprises less than 30%, less than 20%, less than 10%, less than 5%, less than 1%, less than 0.5%, less than 0.25%, less than 0.1%, less than 0.05%, less than 0.025%, less than 0.01%, less than 0.005%, less than 0.0025%, less than 0.001% of L-amino acids. In preferred embodiments, the percentage of L-amino acid is relative to the corresponding D-amino acid. By way of example, a racemic mixture of L-amino acid and D-amino acid contains 50% L-amino acid.

In some embodiments, the composition is essentially free of detergent. For example, the composition comprises, less than about 30 wt %, less than about 20 wt %, less than about 10 wt %, less than about 5 wt %, less than about 1 wt %, less than about 0.5 wt %, less than about 0.25 wt %, less than about 0.1 wt %, less than about 0.05 wt %, less than about 0.025 wt %, less than about 0.01 wt %, less than about 0.005 wt %, less than about 0.0025 wt %, less than about 0.001 wt %, or less, of a detergent. In other embodiments, the composition comprises, relative to the overall composition, less than about 30 wt %, less than 20 wt %, less than 10 wt %, less than 5 wt %, less than 1 wt %, less than 0.5 wt %, less than 0.25 wt %, less than 0.1 wt %, less than 0.05 wt %, less than 0.025 wt %, less than 0.01 wt %, less than 0.005 wt %, less than 0.0025 wt %, less than 0.001 wt % of a detergent. Many times in formulations containing detergents, e.g., surfactants, the surfactant will interact with the active agent, ere the D-amino acid, which could greatly affect the agent's efficacy. In some embodiments, it can be necessary to screen agents effectiveness relative to anionic surfactants, cationic surfactants, non-ionic surfactants and zwitter ionic surfactants as a screening to determine if the presence of the surfactant type alters the efficacy. Reducing or eliminating detergents, can increase the efficacy of the compositions and/or reduce formulation complications.

In other embodiments, the composition is essentially free of both detergent and L-amino acids.

### Biofilms

Most bacteria can form complex, matrix-containing multicellular communities known as biofilms (O'Toole et al., Annu Rev. Microbiol. 54:49 (2000); Lopez et al., FEMS Microbiol. Rev. 33:152 (2009); Karatan et al., Microbiol. Mol. Biol. Rev. 73:310 (2009)). Biofilm-associated bacteria are protected from environmental insults, such as antibiotics (Bryers, Biotechnol. Bioeng. 100:1 (2008)). However, as biofilms age, nutrients become limiting, waste products accumulate, and it is advantageous for the biofilm-associated bacteria to return to a planktonic existence (Karatan et al., Microbiol. Mol. Biol. Rev. 73:310 (2009)). Thus, biofilms have a finite lifetime, characterized by eventual disassembly.

Gram-negative bacteria and Gram-positive bacteria, in addition to other unicellular organisms, can produce biofilms. Bacterial biofilms are surface-attached communities of cells that are encased within an extracellular polysaccharide matrix produced by the colonizing cells. Biofilm development occurs by a series of programmed steps, which include initial attachment to a surface, formation of three-dimensional microcolonies, and the subsequent development of a mature biofilm. The more deeply a cell is located within a biofilm (such as, the closer the cell is to the solid surface to which the biofilm is attached to, thus being more shielded and protected by the bulk of the biofilm matrix), the more metabolically inactive the cells are. The consequences of this physiologic variation and gradient create a collection of bacterial communities where there is an efficient system established whereby microorganisms have diverse functional traits. A biofilm also is made up of various and diverse non-cellular components and can include, but are not limited to carbohydrates (simple and complex), lipids, proteins (including polypeptides), and lipid complexes of sugars and proteins (lipopolysaccharides and lipoproteins). A biofilm may include an integrated community of two or more bacteria species (polymicrobic biofilms), or predominantly one specific bacterium.

The biofilm can allow bacteria to exist in a dormant state for a certain amount of time until suitable growth conditions arise thus offering the microorganism a selective advantage to ensure its survival. However, this selection can pose serious threats to human health in that biofilms have been observed to be involved in about 65% of human bacterial infections (Smith, Adv. Drug Deliv. Rev. 57:1539-1550 (2005); Hall-Stoodley et al., Nat. Rev. Microbiol. 2:95-108 (2004)).

As described herein, biofilms can also affect a wide variety of biological, medical, commercial, industrial, and processing operations.

### Biofilm-Forming Bacteria

The methods described herein can be used to prevent or delay the formation of, and/or treat, biofilms. In exemplary methods, the biofilms are formed by biofilm-forming bacteria. The bacteria can be a gram negative bacterial species or a gram positive bacterial species. Nonlimiting examples of such bacteria include a member of the genus Actinobacillus (such as Actinobacillus actinomycetemcomitans), a member of the genus Acinetobacter (such as Acinetobacter baumannii), a member of the genus Aeromonas, a member of the genus Bordetella (such as Bordetella pertussis, Bordetella bronchiseptica, or Bordetella parapertussis), a member of the genus Brevibacillus, a member of the genus Brucella, a member of the genus Bacteroides (such as Bacteroides fragilis), a member of the genus Burkholderia (such as Burkholderia cepacia or Burkholderia pseudomallei), a member of the genus Borelia (such as Borelia burgdorferi), a member of the genus Bacillus (such as Bacillus anthracis or Bacillus subtilis), a member of the genus Campylobacter (such as Campylobacter jejuni), a member of the genus Capnocytophaga, a member of the genus Cardiobacterium (such as Cardiobacterium hominis), a member of the genus Citrobacter, a member of the genus Clostridium (such as Clostridium tetani or Clostridium difficile), a member of the genus Chlamydia (such as Chlamydia trachomatis, Chlamydia pneumoniae, or Chlamydia psiffaci), a member of the genus Eikenella (such as Eikenella corrodens), a member of the genus Enterobacter, a member of the genus Escherichia (such as Escherichia coli), a member of the genus Francisella (such as Francisella tularensis), a member of the genus Fusobacterium, a member of the genus Flavobacterium, a member of the genus Haemophilus (such as Haemophilus ducreyi or Haemophilus influenzae), a member of the genus Helicobacter (such as Helicobacter pylori), a member of the genus Kingella (such as Kingella kingae), a member of the genus Klebsiella (such as Klebsiella pneumoniae), a member of the genus Legionella (such as Legionella pneumophila), a member of the genus Listeria (such as Listeria monocytogenes), a member of the genus Leptospirae, a member of the genus Moraxella (such as Moraxella catarrhalis), a member of the genus Morganella, a member of the genus Mycoplasma (such as Mycoplasma hominis or Mycoplasma pneumoniae), a member of the genus Mycobacterium (such as Mycobacterium tuberculosis or Mycobacterium leprae), a member of the genus Neisseria (such as Neisseria gonorrhoeae or Neisseria meningitidis), a member of the genus Pasteurella (such as Pasteurella multocida), a member of the genus Proteus (such as Proteus vulgaris or Proteus mirablis), a member of the genus Prevotella, a member of the genus Plesiomonas (such as Plesiomonas shigelloides), a member of the genus Pseudomonas (such as Pseudomonas aeruginosa), a member of the genus Providencia, a member of the genus Rickettsia (such as Rickettsia rickettsii or Rickettsia typhi), a member of the genus Stenotrophomonas (such as Stenotrophomonas maltophila), a member of the genus Staphylococcus (such as Staphylococcus aureus or Staphylococcus epidermidis), a member of the genus Streptococcus (such as Streptococcus viridans, Streptococcus pyogenes (group A), Streptococcus agalactiae (group B), Streptococcus bovis, or Streptococcus pneumoniae), a member of the genus Streptomyces (such as Streptomyces hygroscopicus), a member of the genus Salmonella (such as Salmonella enteriditis, Salmonella typhi, or Salmonella typhimurium), a member of the genus Serratia (such as Serratia marcescens), a member of the genus Shigella, a member of the genus Spirillum (such as Spirillum minus), a member of the genus Treponema (such as Treponema pallidum), a member of the genus Veillonella, a member of the genus Vibrio (such as Vibrio cholerae, Vibrio parahaemolyticus, or Vibrio vulnificus), a member of the genus Yersinia (such as Yersinia enterocolitica, Yersinia pestis, or Yersinia pseudotuberculosis), and a member of the genus Xanthomonas (such as Xanthomonas maltophilia).

Specifically, Bacillus subtilis forms architecturally complex communities on semi-solid surfaces and thick pellicles at the air/liquid interface of standing cultures (Lopez et al., FEMS Microbiol. Rev. 33:152 (2009); Aguilar et al., Curr. Opin. Microbiol. 10:638 (2007); Vlamakis et al., Genes Dev. 22:945 (2008); Branda et al., Proc. Natl. Acad. Sci. USA 98:11621 (2001)). B. subtilis biofilms consist of long chains of cells held together by an extracellular matrix consisting of an exopolysaccharide and amyloid fibers composed of the protein TasA (Branda et al., Proc. Natl. Acad. Sci. USA 98:11621 (2001); Branda et al., Mol. Microbiol. 59:1229 (2006); Romero et al., Proc. Natl. Acad. Sci. USA (2010, in press)). The exopolysaccharide is produced by enzymes encoded by the epsA-O operon (“eps operon”) and the TasA protein is encoded by the promoter-distal gene of the yqxM-sipW-tasA operon (“yqxM operon”) (Chu et al., Mol. Microbiol. 59:1216 (2006)).

Biofilm-producing bacteria, e.g., a species described herein, can be found in a live subject, in vitro, or on a surface, as described herein.

### Applications/Formulations

In instances where a D-amino acid is to be administered to a subject, the D-amino acids described herein can be incorporated into pharmaceutical compositions. The D-amino acids can be incorporated into pharmaceutical compositions as pharmaceutically acceptable salts, esters. or derivatives of the D-amino acids. Such compositions typically include a D-amino acid and a pharmaceutically acceptable carrier. As used herein, a “pharmaceutically acceptable carrier” means a carrier that can be administered to a subject together with a D-amino acid described herein, which does not destroy the pharmacological activity thereof. Pharmaceutically acceptable carriers include, e.g., solvents, binders, dispersion media, coatings, preservatives, colorants, isotonic and absorption delaying agents, and the like, compatible with pharmaceutical administration. Supplementary active compounds can also be incorporated into the compositions.

The term “pharmaceutically acceptable salts” includes, but is not limited to, water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

The D-amino acids may also be in the form of esters or derivatives. Examples of suitable esters include formates, acetates, propionates, butyrates, isobutyrates, pentanoates, crotonates, and benzoates. Some pharmaceutically acceptable derivatives include a chemical group which increases aqueous solubility.

Non-limiting examples of pharmaceutically acceptable carriers that can be used include poly(ethylene-co-vinyl acetate), PVA, partially hydrolyzed poly(ethylene-co-vinyl acetate), poly(ethylene-co-vinyl acetate-co-vinyl alcohol), a cross-linked poly(ethylene-co-vinyl acetate), a cross-linked partially hydrolyzed poly(ethylene-co-vinyl acetate), a cross-linked poly(ethylene-co-vinyl acetate-co-vinyl alcohol), poly-D,L-lactic acid, poly-L-lactic acid, polyglycolic acid, PGA, copolymers of lactic acid and glycolic acid (PLGA), polycaprolactone, polyvalerolactone, poly (anhydrides), copolymers of polycaprolactone with polyethylene glycol, copolymers of polylactic acid with polyethylene glycol, polyethylene glycol; and combinations and blends thereof.

Other carriers include, e.g., an aqueous gelatin, an aqueous protein, a polymeric carrier, a cross-linking agent, or a combination thereof. In other instances, the carrier is a matrix. In yet another instances, the carrier includes water, a pharmaceutically acceptable buffer salt, a pharmaceutically acceptable buffer solution, a pharmaceutically acceptable antioxidant, ascorbic acid, one or more low molecular weight pharmaceutically acceptable polypeptides, a peptide comprising about 2 to about 10 amino acid residues, one or more pharmaceutically acceptable proteins, one or more pharmaceutically acceptable amino acids, an essential-to-human amino acid, one or more pharmaceutically acceptable carbohydrates, one or more pharmaceutically acceptable carbohydrate-derived materials, a non-reducing sugar, glucose, sucrose, sorbitol, trehalose, mannitol, maltodextrin, dextrins, cyclodextrin, a pharmaceutically acceptable chelating agent, EDTA, DTPA, a chelating agent for a divalent metal ion, a chelating agent for a trivalent metal ion, glutathione, pharmaceutically acceptable nonspecific serum albumin, and/or combinations thereof.

A pharmaceutical composition containing a D-amino acid can be formulated to be compatible with its intended route of administration as known by those of ordinary skill in the art. Nonlimiting examples of routes of administration include parenteral, e.g., intravenous, intradermal, subcutaneous, oral (e.g., inhalation), transdermal (topical), transmucosal, vaginal and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition can be sterile and can be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be desirable to include isotonic agents, for example, sugars, polyalcohols such as mannitol, sorbitol, or sodium chloride in the composition. Prolonged absorption of the injectable compositions can be accomplished by including in the composition an agent that delays absorption, for example, aluminum monostearate and gelatin (see, e.g., Remington: The Science and Practice of Pharmacy, 21st edition, Lippincott Williams & Wilkins, Gennaro, ed. (2006)).

Sterile injectable solutions can be prepared by incorporating a D-amino acid in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation include, without limitation, vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, a D-amino acid can be incorporated with excipients and used in the form of tablets, pills, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, a D-amino acid can be delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, but are not limited to, for example, for transmucosal administration, detergents, bile salts, and fusidic acid derivatives. Transmucosal administration can be accomplished through the use of nasal sprays or suppositories. For transdermal administration, the active compounds are formulated into, e.g., ointments, salves, gels, or creams as generally known in the art.

For treatment of acute or chronic wounds, a D-amino acid can be formulated as a dressing, a wash solution, gel, or a synthetic tissue.

A biofilm can form on an oral surface (such as teeth, tongue, back of throat, and the like). These biofilms can be associated with day-to-day bacterial activity of natural flora located in such environments, but can also be associated with oral-related disease(s), such as periodontal disease (for example, gingivitis or periodontitis), breath malodor, or dental caries. By example, periodontitis, a common form of periodontal disease, is believed to be caused by a small group of Gram-negative bacteria present on the tooth root surfaces as biofilms, in particular, Porphyromonas gingivalis, Bacteroides forsythus and Actinobacillus actinomycetemcomitans, with the latter found mostly in cases of juvenile periodontitis. Other bacteria which may be involved in periodontal disease include T. denticola, T. socranskii, F. nucleatum, and P. intermedia, L. acidophilus, L. casei, A. viscosus, S. sobrinus, S. sanguis, S. viridans, and S. mutans. Application of D-amino acid onto such oral surfaces can inhibit or prevent bacterial biofilm formation. Generally, application onto such oral surfaces will be via a product which, in the ordinary course of usage, is not intentionally swallowed for purposes of systemic administration but is rather retained in the oral cavity for a time sufficient to contact substantially all of the dental surfaces and/or oral tissues. The D-amino acid for use on oral surfaces can be formulated as a gum, paste (such as toothpaste), which can then be directly applied to the biofilm of such a surface in a subject. The paste formulation can further comprise an abrasive. A D-amino acid can also exist as a gel formulation or in liquid formulation. For example, the D-amino acid can be formulated as a mouthwash that can directly come into contact with the biofilm on the oral surface of a subject. Additionally, a D-amino acid can be formulated as a polymer film or platelet (e.g., as a slow-release formulation) for treating or preventing oral conditions. In one embodiment, the D-amino acids of the present invention may be used for adjunctive antimicrobial therapy for periodontitis and applied directly to a tooth or between teeth in the form of a chip. The oral care compositions of the present invention may be in various forms including therapeutic rinses, especially mouth rinses; dentifrices such as toothpastes, tooth gels, and tooth powders; non-abrasive gels; mouth sprays; mousse; foams; chewing gums, lozenges and breath mints; drinking water additives; dental solutions and irrigation fluids; and dental implements such as dental floss and tape. The dental implement can be impregnated fibers including dental floss or tape, chips, strips, films and polymer fibers.

For example, an oral composition can contain from about 0.01% to about 15% by weight, e.g., 0.01% to 15% by weight, based on the total weight of the composition, of one or more D-amino acid, and orally tolerable adjuvants. One nonlimiting example of an oral composition includes 10% by weight sorbitol, 10% by weight glycerol, 15% by weight ethanol, 15% by weight propylene glycol, 0.5% by weight sodium lauryl sulfate, 0.25% by weight sodium methylcocyl taurate, 0.25% by weight polyoxypropylene/polyoxyethylene block copolymer, 0.10% by weight peppermint flavouring, 0.1 to 0.5% by weight of one or more D-amino acid, and 48.6% by weight water.

An oral composition can be, for example, in the form of a gel, a paste, a cream or an aqueous preparation (mouthwash). The oral composition can also comprise compounds that release fluoride ions which are effective against the formation of caries, for example inorganic fluoride salts, e.g. sodium, potassium, ammonium or calcium fluoride, or organic fluoride salts, e.g. amine fluorides, which are known under the trade name OLAFLUOR. Oral compositions can further comprise compounds known in the art to be “orally acceptable carriers,” which as used herein means conventional additives in oral care compositions including but not limited to fluoride ion sources, anti-calculus or anti-tartar agents, buffers, abrasives such as silica, bleaching agents such as peroxide sources, alkali metal bicarbonate salts, thickening materials, humectants, water, surfactants, titanium dioxide, flavor system, sweetening agents, xylitol, coloring agents, and mixtures thereof. Such materials are well known in the art and are readily chosen by one skilled in the art based on the physical, aesthetic and performance properties desired for the compositions being prepared. These carriers may be included at levels typically from about 50% to about 99%, preferably from about 70% to about 98%, and more preferably from about 90% to about 95%, by weight of the oral composition. The choice of a carrier to be used is basically determined by the way the composition is to be introduced into the oral cavity. In one preferred embodiment, the oral compositions are in the form of dentifrices, such as toothpastes, tooth gels and tooth powders. Components of such toothpaste and tooth gels generally include one or more of a dental abrasive (from about 6% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion) and an anticalculus agent (from about 0.1% to about 13%). Tooth powders contain substantially all non-liquid components. Other preferred oral care compositions are liquid products, including mouthwashes or rinses, mouth sprays, dental solutions and irrigation fluids. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion) and an anticalculus agent (from about 0.1% to about 3%). Components of dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

The pharmaceutical compositions containing a D-amino acid can also be prepared in the form of suppositories (e.g., with conventional suppository bases such as cocoa butter and other glycerides) or retention enemas for rectal delivery.

In some embodiments, the composition is essentially free of detergent. In some instances, a detergent can contribute to the toxicity of a composition. For example, the composition comprises less than about 30%, less than about 20%, less than about 10%, less than about 5%, less than about 1%, less than about 0.5%, less than about 0.25%, less than about 0.1%, less than about 0.05%, less than about 0.025%, less than about 0.01%, less than about 0.005%, less than about 0.0025%, less than about 0.001%, or less, of a detergent, e.g., less than 30%, less than 20%, less than 10%, less than 5%, less than 1%, less than 0.5%, less than 0.25%, less than about 0.1%, less than 0.05%, less than 0.025%, less than 0.01%, less than 0.005%, less than about 0.0025%, less than 0.001%, of a detergent.

Some pharmaceutical compositions can be prepared with a carrier that protects the D-amino acid against rapid elimination from the body, such as a controlled release formulation, including implants and microencapsulated delivery systems (as described, e.g., in Tan et al., Pharm. Res. 24:2297-2308, 2007). Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially (e.g., from Alza Corp., Mountain View, Calif.). Liposomal suspensions (including liposomes targeted to particular cells with monoclonal antibodies to cell surface antigens) can also be used as pharmaceutically acceptable carriers. These can be prepared according to methods known to those skilled in the art, e.g., as described in U.S. Pat. No. 4,522,811.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. While compounds that exhibit toxic side effects can be used, care should be taken to design a delivery system that targets such compounds to the site of affected tissue in order to minimize potential damage to normal cells and, thereby, reduce side effects.

The data obtained from the cell culture assays and animal studies can be used in formulating a range of dosage for use in humans. The dosage of such compounds lies generally within a range of circulating concentrations that include the ED₅₀ with little or no toxicity. The dosage can vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography. Information for preparing and testing such compositions are known in the art (see, e.g., Remington: The Science and Practice of Pharmacy, 21st edition, Lippincott Williams & Wilkins, Gennaro, ed. (2006)).

In some instances, about 0.0005 μM D-amino acid to about 50 μM D-amino acid is administered, e.g., about 0.001 μM D-amino acid to about 25 μM D-amino acid, about 0.002 μM D-amino acid to about 10 μM D-amino acid, about 0.003 μM D-amino acid to about 5 μM D-amino acid, about 0.004 μM D-amino acid to about 1 μM D-amino acid, about 0.005 μM D-amino acid to about 0.5 μM D-amino acid, about 0.01 μM D-amino acid to about 0.1 μM D-amino acid, or about 0.02 μM D-amino acid to about 0.1 μM D-amino acid, e.g., 0.0005 μM D-amino acid to 50 μM D-amino acid is administered, 0.001 μM D-amino acid to 25 μM D-amino acid, 0.002 μM D-amino acid to 10 μM D-amino acid, 0.003 μM D-amino acid to 5 μM D-amino acid, 0.004 μM D-amino acid to 1 μM D-amino acid, 0.005 μM D-amino acid to 0.5 μM D-amino acid, 0.01 μM D-amino acid to 0.1 μM D-amino acid, or 0.02 μM D-amino acid to 0.1 μM D-amino acid. Preferably, a D-amino acid is administered at nanomolar concentrations, e.g., at about 5 nM, at about 10 nM, at about 15 nM, at about 20 nM, at about 25 nM, at about 30 nM, at about 50 nM, or more, or preferably at 5 nM, at 10 nM, at 15 nM, at 20 nM, at 25 nM, at 30 nM, OR at 50 Nm.

In other instances, a therapeutically effective amount or dosage of a D-amino acid can range from about 0.001 mg/kg body weight to about 100 mg/kg body weight, e.g., from about 0.01 mg/kg body weight to about 50 mg/kg body weight, from about 0.025 mg/kg body weight to about 25 mg/kg body weight, from about 0.1 mg/kg body weight to about 20 mg/kg body weight, from about 0.25 mg/kg body weight to about 20 mg/kg body weight, from about 0.5 mg/kg body weight to about 20 mg/kg body weight, from about 0.5 mg/kg body weight to about 10 mg/kg body weight, from about 1 mg/kg body weight to about 10 mg/kg body weight, or about 5 mg/kg body weight, or preferably 0.001 mg/kg body weight to 100 mg/kg body weight, e.g., from 0.01 mg/kg body weight to 50 mg/kg body weight, from 0.025 mg/kg body weight to 25 mg/kg body weight, from 0.1 mg/kg body weight to 20 mg/kg body weight, from 0.25 mg/kg body weight to 20 mg/kg body weight, from 0.5 mg/kg body weight to 20 mg/kg body weight, from 0.5 mg/kg body weight to 10 mg/kg body weight, from 1 mg/kg body weight to 10 mg/kg body weight, or 5 mg/kg body weight.

A physician will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a D-amino acid can include a single treatment or a series of treatments. In one example, a subject is treated with a D-amino acid in the range of between about 0.06 mg to about 120 mg, one time per week for between about 1 to 10 weeks, alternatively between 2 to 8 weeks, between about 3 to 7 weeks, or for about 4, 5, or 6 weeks, or preferably between 0.06 mg to 120 mg, one time per week for between 1 to 10 weeks, alternatively between 2 to 8 weeks, between 3 to 7 weeks, or for 4, 5, or 6 weeks. It will also be appreciated that the effective dosage of a D-amino acid used for treatment may increase or decrease over the course of a particular treatment.

The pharmaceutical compositions can be included in a container, pack, or dispenser together with instructions for administration. A person of ordinary skill in the art will appreciate that the pharmaceutical compositions described herein can be formulated as single-dose vials.

Treatment of a subject with a therapeutically effective amount of a D-amino acid-containing pharmaceutical composition described herein can be a single treatment, continuous treatment, or a series of treatments divided into multiple doses. The treatment can include a single administration, continuous administration, or periodic administration over one or more years. Chronic, long-term administration can be indicated in some cases. Generally, each formulation is administered in an amount sufficient to suppress or reduce or eliminate a deleterious effect or a symptom of a biofilm-related disorder or condition described herein.

D-amino acids are suitable as antibiofilm active substances in personal care preparations, for example shampoos, bath additives, hair care preparations, liquid and solid soaps (based on synthetic surfactants and salts of saturated and/or unsaturated fatty acids), lotions and creams, deodorants, other aqueous or alcoholic solutions, e.g. cleansing solutions for the skin, moist cleaning cloths, oils or powders. Propionibacterium acnes, which is the predominant microorganism occurring in acne, may reside in biofilms. Thus, D-amino acids are particularly suitable for personal care compositions for use in controlling acne. The invention accordingly relates also to personal care preparations comprising one or more D-amino acids described herein and cosmetically tolerable carriers or adjuvants.

The D-amino acids described herein are also suitable for imparting antibiofilm properties to a range of formulations used in personal care. Personal care preparations can contain from about 0.01% to about 15% by weight, for example, from about 0.1% to about 10% by weight, or 0.01% to 15% by weight, for example, from 0.1% to 10% by weight, based on the total weight of the preparation, of one or more D-amino acids, and cosmetically tolerable adjuvants. Depending on the form of the personal care preparation, such preparation can include, in addition to one or more D-amino acids, further constituents, for example sequestering agents, colourings, perfume oils, thickening or solidifying agents (consistency regulators), emollients, UV-absorbers, skin protective agents, antioxidants, additives that improve the mechanical properties, such as dicarboxylic acids and/or aluminium, zinc, calcium or magnesium salts of C₁₄-C₂₂ fatty acids, and, optionally, preservatives.

In one embodiment, the anti-acne composition comprising D-amino acids can further comprise at least one antimicrobial agent. Preferably, the antimicrobial agent is an antibiotic. The antibiotic may be selected from the group consisting of tobramycin, clindamycin, ciprofloxacin, tetracyclines, rifampin, triclosan, oxfloxacin, macrolides, penicillins, cephalosporins, amoxicillin/clavulante, quinupristin/dalfopristin, amoxicillin/sulbactum, metronidazole, fluoroquinolones, quinolones, ketolides, or aminoglycosides. The present invention provides a method for controlling acne, comprising administering to a subject afflicted with acne an effective amount of an anti-acne composition comprising one or more D-amino acids, wherein the amount of the D-amino acids in the anti-acne composition is sufficient to prevent, reduce, inhibit or remove a biofilm.

Personal care preparations can be in the form of a water-in-oil or oil-in-water emulsion, an alcoholic or alcohol-containing formulation, a vesicular dispersion of an ionic or non-ionic ampiphilic lipid, a gel, a solid stick or an aerosol formulation. As a water-in-oil or oil-in-water emulsion, the cosmetically tolerable adjuvant contains preferably from about 5% to about 50% of an oil phase, from about 5% to about 20% of an emulsifier and from about 30% to 90% water, or 5% to 50% of an oil phase, from 5% to 20% of an emulsifier and from 30% to 90% water. The oil phase can comprise any oil suitable for cosmetic formulations, for example one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

Cosmetic formulations described herein are used in various fields. Such preparations include, without limitation, for example:
skin-care preparations, e.g. skin-washing and cleansing preparations in the form of tablet-form or liquid soaps, synthetic detergents or washing pastes,
bath preparations, e.g. liquid (foam baths, milks, shower preparations) or solid bath preparations, e.g. bath cubes and bath salts;
skin-care preparations, e.g. skin emulsions, multi-emulsions or skin oils;
cosmetic personal care preparations, e.g. facial make-up in the form of day creams or powder creams, face powder (loose or pressed), rouge or cream make-up, eye-care preparations, e.g. eye shadow preparations, mascaras, eyeliners, eye creams or eye-fix creams; lip-care preparations, e.g. lipsticks, lip gloss, lip contour pencils, nail-care preparations, such as nail varnish, nail varnish removers, nail hardeners or cuticle removers;
intimate hygiene preparations, e.g. intimate washing lotions or intimate sprays;
foot-care preparations, e.g. foot baths, foot powders, foot creams or foot balsams, special deodorants and antiperspirants or callus-removing preparations;
light-protective preparations, such as sun milks, lotions, creams or oils, sun-blocks or tropicals, pre-tanning preparations or after-sun preparations;
skin-tanning preparations, e.g. self-tanning creams;
depigmenting preparations, e.g. preparations for bleaching the skin or skin-lightening preparations;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or aftershave lotions;
fragrance preparations, e.g. fragrances (eau de Cologne, eau de toilette, eau de parfum, parfum de toilette, perfume), perfume oils or perfume creams;
dental care, denture-care and mouth-care preparations, e.g. toothpastes, gel toothpastes, tooth powders, mouthwash concentrates, anti-plaque mouthwashes, denture cleaners or denture fixatives;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidising dyes, or natural hair colorants, such as henna or camomile.

The following represent nonlimiting examples of various formulations that can be prepared containing one or more D-amino acids. A wide variety of similar formulations are known in the art into which one or more D-amino acids can readily be incorporated at various concentrations.

An exemplary soap has, for example, the following composition: 0.01 to 5% by weight of one or more D-amino acids, 0.3 to 1% by weight titanium dioxide, 1 to 10% by weight stearic acid, soap base ad 100%, e.g. a sodium salt of tallow fatty acid or coconut fatty acid, or glycerol.

An exemplary shampoo has, for example, the following composition: 0.01 to 5% by weight of one or more D-amino acids, 12.0% by weight sodium laureth-2-sulfate, 4.0% by weight cocamidopropyl betaine, 3.0% by weight NaCl and water ad 100%.

An exemplary deodorant has, for example, the following composition: 0.01 to 5% by weight of one or more D-amino acids, 60% by weight ethanol, 0.3% by weight perfume oil, and water ad 100%.

In some instances, a D-amino acid pharmaceutical composition is administered to prevent or reduce biofilm formation on a biologically relevant surface or substrate. These surfaces include, but are not limited to, an epithelial or mucosal surface of the respiratory tract, lungs, the oral cavity, the alimentary and vaginal tracts, in the ear or the surface of the eye, and the urinary tract. For example, a biofilm can affect the surface of a lung (such as the lung of a subject with pneumonia, cystic fibrosis, or COPD), such as epithelial cells of the lung.

In certain embodiments, the surface is a biologically relevant surface is a surface that is likely to contact a biological fluid, e.g., a liquid component of a subject such as blood, serum, sputum, lacrimal secretions, semen, urine, vaginal secretions, and tissue samples and the like. The biologically relevant surface can be a component of a medical device, instrument, or implant. Nonlimiting examples include clamps, forceps, scissors, skin hooks, tubing (such as endotracheal or gastrointestinal tubes), needles, retractors, scalers, drills, chisels, rasps, saws, catheters including indwelling catheter (such as urinary catheters, vascular catheters, peritoneal dialysis catheter, central venous catheters), catheter components (such as needles, Leur-Lok connectors, needleless connectors), orthopedic devices, artificial heart valves, prosthetic joints, voice prostheses, stents, shunts, pacemakers, surgical pins, respirators, ventilators, and endoscopes. The present invention is particularly well-suited to substantially reduce the risk of biofilm accumulation on the surfaces of a medical device adapted for prolonged term implantation, wherein the medical device is intended to remain implanted for a relatively long period of from about 30 days to about 12 months or longer, and the resultant likelihood of premature failure of the device due to encrustation and occlusion by such biofilm. However, such encrustation may occur on medical devices after shorter periods of time, such as 30 days or less, as well, which would also be understood to be devices for prolonged term implantation. For example, in certain embodiments, a medical device utilized for a prolonged period of time may implanted for a period longer than 24 hours, such as a week.

In certain instances, a subject can be administered a D-amino acid prior to, during, or after implantation/insertion of a medical device, catheter, stent, prosthesis, and the like, or application of a wound dressing. In some instances, the wound dressing includes an antimicrobial, such as silver. Treatment before or after implantation can take place immediately before or after the implantation or several hours before or after implantation, or at a time or times that the skilled physician deems appropriate.

A D-amino acid can be applied to a surface by any known means, such as by covering, coating, contacting, associating with, filling, or loading the surface with a therapeutic amount of a D-amino acid. In specific examples, a D-amino acid is directly affixing to a surface by either spraying the surface with a polymer/D-amino acid film, by dipping the surface into a polymer/D-amino acid solution, or by other covalent or noncovalent means. In other instances, the surface is coated with a substance (such as a hydrogel) that absorbs the D-amino acid.

The composition can be a coating or a film. When applied as a part of a film or coating, one or more D-amino acid described herein can be part of a composition which also comprises a binder. The binder may be any polymer or oligomer compatible with the present antibiofilms. The binder may be in the form of a polymer or oligomer prior to preparation of the antibiofilm composition, or may form by polymerization during or after preparation, including after application to the substrate. In certain applications, such as certain coating applications, it will be desirable to crosslink the oligomer or polymer of the antibiofilm composition after application. The term “binder” as used herein includes materials such as glycols, oils, waxes and surfactants commercially used in the pharmaceutical and personal care industries. It is preferred that materials that are Generally Regarded as Safe (G.R.A.S.) be used.

When the composition is a thermoplastic film which is applied to a surface, for example, by the use of an adhesive or by melt applications including calendaring and co-extrusion, the binder is the thermoplastic polymer matrix used to prepare the film. When the composition is a coating, it may be applied as a liquid solution or suspension, a paste, gel, oil or the coating composition may be a solid, for example a powder coating which is subsequently cured by heat, UV light or other method.

As the composition of the invention may be a coating or a film, the binder can be comprised of any polymer used in coating formulations or film preparation. For example, the binder is a thermoset, thermoplastic, elastomeric, inherently crosslinked or crosslinked polymer. Thermoset, thermoplastic, elastomeric, inherently crosslinked or crosslinked polymers include polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, silicon containing and carbamate polymers, fluorinated polymers, crosslinkable acrylic resins derived from substituted acrylic esters, e.g. from epoxy acrylates, urethane acrylates or polyester acrylates. The polymers may also be blends and copolymers of the preceding chemistries.

Biocompatible coating polymers, such as, poly[-alkoxyalkanoate-co-3-hydroxyalkenoate] (PHAE) polyesters, Geiger et. al. Polymer Bulletin 52, 65-70 (2004), can also serve as binders in the present invention. Alkyd resins, polyesters, polyurethanes, epoxy resins, silicone containing polymers, polyacrylates, polyacrylamides, fluorinated polymers and polymers of vinyl acetate, vinyl alcohol and vinyl amine are non-limiting examples of common coating binders useful in the present invention. Other known coating binders are part of the present disclosure.

Coatings can be crosslinked with, for example, melamine resins, urea resins, isocyanates, isocyanurates, polyisocyanates, epoxy resins, anhydrides, poly acids and amines, with or without accelerators. The compositions described herein can be, for example, a coating applied to a surface which is exposed to conditions favorable for bioaccumulation. The presence of one or more D-amino acids described herein in said coating can prevent the adherence of organisms to the surface.

The coating may be solvent borne or aqueous. Aqueous coatings are typically considered more environmentally friendly. In some examples, the coating can be an aqueous dispersion of one or more D-amino acids described herein and a binder or a water based coating or paint. For example, the coating can comprise an aqueous dispersion of one or more D-amino acids and an acrylic, methacrylic or acrylamide polymers or co-polymers or a poly[-alkoxyalkanoate-co-3-hydroxyalkenoate]polyester.

In some instances, the coating composition can be applied to a surface by any conventional means including spin coating, dip coating, spray coating, draw down, or by brush, roller or other applicator. A drying or curing period can be performed.

Coating or film thickness can vary depending on the application and can readily be determined by one skilled in the art after limited testing.

In some instances, a composition described herein can be in the form of a protective laminate film. Such a film can comprise thermoset, thermoplastic, elastomeric, or crosslinked polymers. Examples of such polymers include, but are not limited to, polyolefin, polyamide, polyurethane, polyacrylate, polyacrylamide, polycarbonate, polystyrene, polyvinyl acetates, polyvinyl alcohols, polyester, halogenated vinyl polymers such as PVC, natural and synthetic rubbers, alkyd resins, epoxy resins, unsaturated polyesters, unsaturated polyamides, polyimides, fluorinated polymers, silicon containing and carbamate polymers. The polymers can also be blends and copolymers of the preceding chemistries.

When a composition described herein is a preformed film, it can be applied to a surface by, for example, the use of an adhesive, or co-extruded onto the surface. It can also be mechanically affixed via fasteners which may require the use of a sealant or caulk wherein the esters of the instant invention may also be advantageously employed. A plastic film can also be applied with heat which includes calendaring, melt applications and shrink wrapping.

Given the wide array of applications for the D-amino acids described herein, a D-amino acid-containing composition can include other additives such as antioxidants, UV absorbers, hindered amines, phosphites or phosphonites, benzofuran-2-ones, thiosynergists, polyamide stabilizers, metal stearates, nucleating agents, fillers, reinforcing agents, lubricants, emulsifiers, dyes, pigments, dispersants, other optical brighteners, flame retardants, antistatic agents, blowing agents and the like, such as the materials listed below, or mixtures thereof.

Medical devices prepared from plastic can incorporate a D-amino acid during the forming, e.g., molding, process. Plastic-based medical devices that benefit from the present method include, but are not limited to, plastics articles used in the field of medicine, e.g. dressing materials, syringes, catheters etc., so-called “medical devices”, gloves and mattresses. Exemplary of such plastics are polypropylene, polyethylene, PVC, POM, polysulfones, polyethersulfones, polystyrenics, polyamides, polyurethanes, polyesters, polycarbonate, polyacrylics and methacrylics, polybutadienes, thermoplastic polyolefins, ionomers, unsaturated polyesters and blends of polymer resins including ABS, SAN and PC/ABS.

The D-amino acids, especially in low concentrations, can be safely used even in applications where ingestion is possible, such as reusable water bottles or drinking fountains where a biofilm may develop. The surfaces of such water transport devices can be rinsed with a formulation containing one or more D-amino acids described herein, or low levels of one or more D-amino acids can be introduced into the water that passes through the containers of conduits. For example, about 0.0001% or less or up to about 1%, typically less than about 0.1% by weight of one or more D-amino acids may be introduced into such water. Given the high activity of the instant D-amino acids, very small amounts are effective in many circumstances and concentrations of about 0.000001% to about 0.1%, for example, about 0.000001% to about 0.01%, or about 0.000001% to about 0.001%, or 0.000001% to 0.1%, 0.000001% to 0.01%, or 0.000001% to 0.001%, can be used in such applications.

When used in a coating or film, small amounts of one or more D-amino acids can be present for short term use, for example, one use, seasonal or disposable items, especially those applications which involve possible human contact, splints, catheters, tubing, dental equipment etc. In general, about 0.001% or less up to about 5%, for example up to about 3% or about 2%, or preferably 0.001% or less up to 5%, up to 3% or 2% by weight of one or more amino acids may be used in such coatings or films. Given the high activity of the instant D-amino acids, very small amounts are effective in many circumstances and concentrations of about 0.0001% to about 1%, for example, about 0.0001% to about 0.5%, or about 0.0001% to about 0.01% can be used in coating applications, or preferably 0.0001% to 1%, 0.0001% to 0.5%, or 0.0001% to 0.01% by weight of one or more D-amino acids.

For incorporation into a molded plastic article, about 0.00001% to about 10% of one or more D-amino acids can be used, for example about 0.0001% to about 3%, for example about 0.001% up to about 1% one or more D-amino acids can be used, or preferably, 0.00001% to 10%, 0.0001% to 3 0.001% up to 1% by weight one or more D-amino acids can be used. In situations in which the D-amino acids are impregnated into the surface of an already prepared molded article or fiber, the actual amount of a D-amino-acid present at the surface can depend on the substrate material, the formulation of the impregnating composition, and the time and temperature used during the impregnation step. Given the high activity of the instant D-amino acids, very small amounts are effective in many circumstances, and concentrations of about 0.0001% to about 1%, for example, about 0.0001% to about 0.1%, or about 0.0001% to about 0.01% can be used in plastics, or preferably 0.0001% to 1%, 0.0001% to 0.1%, or 0.0001% to 0.01% by weight of one or more amino acids can by used.

Inhibition or reduction in a biofilm by treatment with a D-amino acid can be measured using techniques well established in the art. These techniques enable one to assess bacterial attachment by measuring the staining of the adherent biomass, to view microbes in vivo using microscopy methods; or to monitor cell death in the biofilm in response to toxic agents. Following treatment, the biofilm can be reduced with respect to the surface area covered by the biofilm, thickness, and consistency (for example, the integrity of the biofilm). Non-limiting examples of biofilm assays include microtiter plate biofilm assays, fluorescence-based biofilm assays, static biofilm assays according to Walker et al., Infect. Immun. 73:3693-3701 (2005), air-liquid interface assays, colony biofilm assays, and Kadouri Drip-Fed Biofilm assays (Merritt et al., (2005) Current Protocols in Microbiology 1.B.1.1-1.B.1.17). Such assays can be used to measure the activity of a D-amino acid on the disruption or the inhibition of formation of a biofilm (Lew et al., (2000) Curr. Med. Chem. 7(6):663-72; Werner et al., (2006) Brief Funct. Genomic Proteomic 5(1):32-6).

In other instances, treatment can be assayed by measuring the growth of bacteria and/or can be quantified by measuring the density of a biofilm-forming bacteria in a biological sample. Non-limiting examples of biological samples include blood, serum, sputum, lacrimal secretions, semen, urine, vaginal secretions, and tissue samples. The reduction in the growth of bacteria can also be measured by chest X-rays or by a pulmonary function test (PFT) (for example, spirometry or forced expiratory volume (FEV₁)).

In other situations, the presence or growth of biofilm-producing bacteria can be measured by detecting the presence of antigens of biofilm-producing bacteria in a biological sample, such as those described above. For example, an antibody to S. pneumoniae components can be used to assay colonization/infection in a subject afflicted with a biofilm-related condition or disorder, such as by assaying the presence of Streptococcus antigens in a biological sample. Such antibodies can be generated according to methods well established in the art or can be obtained commercially (for example, from Abcam, Cambridge, Mass.; Cell Sciences Canton, Mass.; Novus Biologicals, Littleton, Colo.; or GeneTex, San Antonio, Tex.).

Appropriate therapies for the treatment of biofilm-related disorders with a D-amino acid can be determined using techniques well established in the art. For example, animal models using mammals can be used to assess the efficacy of treatment with D-amino acids. Non-limiting examples include implanting polymer beads, e.g., polymethylmethacrylate (PMMA) beads loaded with the D-amino acid in rats and assessing their ability to prevent biofilms.) polymethylmethacrylate (PMMA) beads in rats and catheters in rabbits have been used as animal models for biofilm formation for Staph aureus. See, e.g., Anguita-Alonzo et al., ANTIMICROBIAL AGENTS AND CHEMOTHERAPY, July 2007, p. 2594-2596, and Beenken et al. JOURNAL OF BACTERIOLOGY, July 2004, p. 4665-4684, which are hereby incorporated in its entirety by reference.

### Combination Therapy

Biofilms are understood, very generally, to be aggregations of living and dead micro-organisms, especially bacteria, that adhere to living and non-living surfaces, together with their metabolites in the form of extracellular polymeric substances (EPS matrix), e.g. polysaccharides. The activity of antibiofilm substances that normally exhibit a pronounced growth-inhibiting or lethal action with respect to planktonic cells may be greatly reduced with respect to microorganisms that are organized in biofilms, for example because of inadequate penetration of the active substance into the biological matrix.

In some instances, a D-amino acid can be administered alone or in combination with a second agent, e.g., a biocide, an antibiotic, or an antimicrobial agent, to treat a biofilm or to prevent the formation of a biofilm. An antibiotic can be co-administered with the D-amino acid either sequentially or simultaneously. For example, any of the compositions described herein can be formulated to include one or more D-amino acids and one or more second agents.

The antibiotic can be any compound known to one of ordinary skill in the art that can inhibit the growth of, or kill, bacteria. Useful, non-limiting examples of antibiotics include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); glycopeptide antibiotics (such as vancomycin); polypeptide antibiotics (such as bacitracin); macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Pefloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. Such antibiotics are commercially available, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Additional known biocides include biguanide, chlorhexidine, triclosan, chlorine dioxide, and the like.

Useful examples of antimicrobial agents include, but are not limited to, Pyrithiones, especially the zinc complex (ZPT); Octopirox®; Dimethyldimethylol Hydantoin (Glydant®); Methylchloroisothiazolinone/methylisothiazolinone (Kathon CG®); Sodium Sulfite; Sodium Bisulfite; Imidazolidinyl Urea (Germall 1150, Diazolidinyl Urea (Germain IIC); Benzyl Alcohol; 2-Bromo-2-nitropropane-1,3-diol (Bronopol®); Formalin (formaldehyde); Iodo [symbol] pro [symbol] penyl Butylcarbamate (Polyphase P100®); Chloroacetamide; Methanamine; Methyldibromo [symbol] nitrile Glutaronitrile (1,2-Dibromo-2,4-dicyanobutane or Tektamer®); Glutaraldehyde; 5-bro [symbol] mo-5-nitro-1,3-dioxane (Bronidox®); Phenethyl Alcohol; o-Phenylphenol/sodium o-phenyh [symbol] phenol; Sodium Hydroxymethylglycinate (Suttocide A®); Polymethoxy Bicyclic Oxazolidine (Nuosept C®); Dimethoxane; Thimersal; Dichlorobenzyl Alcohol; Captan; Chlorphenenesin; Dichlorophene; Chlorbutanol; Glyceryl Laurate; Halogenated Diphenyl Ethers; 2,4,4′-trichloro-2′-hydroxy-diphenyl ether (Triclosan®. or TCS); 2,2′-dihydroxy-5,5′-dibromo-diphenyl ether; Phenolic Compounds; Phenol; 2-Methyl Phenol; 3-Methyl Phenol; 4-Methyl Phenol; 4-Ethyl Phenol; 2,4-Dimethyl Phenol; 2,5-Dimethyl Phenol; 3,4-Dimethyl Phenol; 2,6-Dimethyl Ph [symbol] enol; 4-n-Propyl Phenol; 4-n-Butyl Phenol; 4-n-Amyl Phenol; 4-tert-Amyl Phenol; 4-n-Hexyl Phenol; 4-n-Heptyl Phenol; Mono- and Poly-Alkyl and Aromatic Halophenols; p-Chloro [symbol] phe [symbol] nol; Methyl p-Chlorophenol; Ethyl p-Chlorophenol; n-Propyl p-Chlorophenol; n-Butyl p-Chlor [symbol] ophenol; n-Amyl p-Chlorophenol; sec-Amyl p-Chlorophenol; Cyclohexyl p-Chloro [symbol] phe [symbol] nol; n-Heptyl p-Chlorophenol; n-Octyl p-Chlorophenol; o-Chlorophenol; Methyl o-Chloro [symbol] phenol; Ethyl o-Chlorophenol; n-Propyl o-Chlorophenol; n-Butyl o-Chlorophenol; n-Amyl o-Chloro [symbol] phenol; tert-Amyl o-Chlorophenol; n-Hexyl o-Chlorophenol; n-Heptyl o-Chlorophenol; o-Ben [symbol] zyl p-Chlorophenol; o-Benxyl-m-methyl p-Chlorophenol; o-Benzyl-m; m-dimethyl p-Chloro [symbol] phenol; o-Phenylethyl p-Chlorophenol; o-Phenylethyl-m-methyl p-Chlorophenol; 3-Methyl p-Chlorophenol; 3,5-Dimethyl p-Chlorophenol; 6-Ethyl-3-methyl p-Chlorophenol; 6-n-Propyl-3-methyl p-Chlorophenol; 6-iso-Propyl-3-methyl p-Chlorophenol; 2-Ethyl-3,5-dimethyl p-Chloro [symbol] phenol; 6-sec-Butyl-3-methyl p-Chlorophenol; 2-iso-Propyl-3,5-dimethyl p-Chlorophenol; 6-Diethylmethyl-3-methyl p-Chlorophenol; 6-iso-Propyl-2-ethyl-3-methyl p-Chlorophenol; 2-sec-Amyl-3,5-dimethyl p-Chlorophenol; 2-Diethylmethyl-3,5-dimethyl p-Chlorophenol; 6-sec-Octyl-3-methyl p-Chlorophenol; p-Chloro-m-cresol: p-Bromophenol; Methyl p-Bromophenol; Ethyl p-Bromophenol; n-Propyl p-Bromophenol; n-Butyl p-Bromophenol; n-Amyl p-Bromo [symbol] phenol; sec-Amyl p-Bromophenol; n-Hexyl p-Bromophenol; Cyclohexyl p-Bromophenol; o-Bromophenol; tert-Amyl o-Bromophenol; n-Hexyl o-Bromophenol; n-Propyl-m,m-Dimethyl o-Bromophenol; 2-Phenyl Phenol; 4-Chloro-2-methyl phenol; 4-Chloro-3-methyl phenol; 4-Chloro-3,5-dimethyl phenol; 2,4-Dichloro-3,5-dimethylphenol; 3,4,5,6-Terabromo-2-methyl [symbol] phenol; 5-Methyl-2-pentylphenol; 4-Isopropyl-3-methylphenol; Para-chloro-meta-xylenol (PCMX); Chlorothymol; Phenoxyethanol; Phenoxyisopropanol; 5-Chloro-2-hydroxydi [symbol] phenyl [symbol] methane; Resorcinol and its Derivatives; Resorcinol; Methyl Resorcinol; Ethyl Resorcinol; n-Propyl Resorcinol; n-Butyl Resorcinol; n-Amyl Resorcinol; n-Hexyl Resorcinol; n-Heptyl Re [symbol] sorcinol; n-Octyl Resorcinol; n-Nonyl Resorcinol; Phenyl Resorcinol; Benzyl Resorcinol; Phe [symbol] nylethyl Resorcinol; Phenylpropyl Resorcinol; p-Chlorobenzyl Resorcinol; 5-Chloro 2,4-Dihy [symbol] droxy [symbol] diphenyl Methane; 4′-Chloro 2,4-Dihydroxydiphenyl Methane; 5-Bromo 2,4-Dihydroxy [symbol] diphenyl Methane; 4′-Bromo 2,4-Dihydroxydiphenyl Methane; Bisphenolic Compounds; 2,2′-Methylene bis-(4-chlorophenol); 2,2′-Methylene bis-(3,4,6-trichlorophenol); 2,2′-Methylene bis-(4-chloro-6-bromophenol); bis(2-hydroxy-3,5-dichlorophenyl)sulfide; bis(2-hydroxy-5-chlo [symbol] ro [symbol] benzyl)sulfide; Benzoic Esters (Parabens); Methylparaben; Propylparaben; Butylparaben; Ethylparaben; Isopropylparaben; Isobutylparaben; Benzylparaben; Sodium Methylpara [symbol] ben; Sodium Propylparaben; Halogenated Carbanilides; 3,4,4′-Trichlorocarbanilides (Triclo [symbol] car [symbol] ban® or TCC); 3-Trifluoromethyl-4,4′-dichlorocarbanilide; 3,3′,4-Trichlorocarbanilide; Chlorohexidine and its digluconate; diacetate and dihydrochloride; Undecenoic acid; thiabendazole, Hexetidine; poly(hexamethylenebiguanide) hydrochloride (Cosmocil®); silver compounds such as organic silver salts it anorganic silver salts, silver chloride including formulations thereof such as JM Acticare® and micronized silver particles.

### Biofilm-Related Disorders

Methods and treatments using D-amino acids include inhibiting or preventing the formation of biofilm, even or especially without inhibiting organism growth, and alos the disruption of a biofilm once formed.

A D-amino acid can be used to treat biofilm-related disorders in a subject by administering to the subject an effective amount of D-amino acid that reduces biofilm formation in the subject. A reduction in bacterial growth is indicative of the reduction in, or inhibition of, biofilm production in the subject.

In some instances, a D-amino acid can inhibit or reduce biofilm formation by diminishing adherence of biofilm-forming bacteria to a surface or by increasing bacterial death. This therapeutic approach can be useful for the treatment of biofilm-related disorders or conditions, or medical device-related infections associated with the formation of microbial biofilms.

Non-limiting examples of biofilm-related disorders include otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), and cystic fibrosis. In one specific example, subjects with cystic fibrosis display an accumulation of biofilm in the lungs and digestive tract. Subjects afflicted with COPD, such as emphysema and chronic bronchitis, display a characteristic inflammation of the airways wherein airflow through such airways, and subsequently out of the lungs, is chronically obstructed.

Biofilm-related disorders can also encompass infections derived from implanted/inserted devices, medical device-related infections, such as infections from biliary stents, orthopedic implant infections, and catheter-related infections (kidney, vascular, peritoneal). An infection can also originate from sites where the integrity of the skin and/or soft tissue has been compromised. Non-limiting examples include dermatitis, ulcers from peripheral vascular disease, a burn injury, and trauma. For example, a Gram-positive bacterium, such as S. pneumoniae, can cause opportunistic infections in such tissues. The ability of S. pneumoniae to infect burn wound sites, e.g., is enhanced due to the breakdown of the skin, burn-related immune defects, and antibiotic selection.Subjects

In some instances, a subject is treated. A subject can be a mammal including, but not limited to, a primate (e.g., a monkey, such as a cynomolgous monkey, a chimpanzee, and a human). A subject can be a non-human animal such as a bird (e.g., a quail, chicken, or turkey), a farm animal (e.g., a cow, goat, horse, pig, or sheep), a pet (e.g., a cat, dog, or guinea pig, rat, or mouse), or laboratory animal (e.g., an animal model for a disorder). Non-limiting representative subjects can be a human infant, a pre-adolescent child, an adolescent, an adult, or a senior/elderly adult.

In some instances, a subject in need of treatment can be one afflicted with one or more of the infections or disorders described herein. In some instances, the subject is at risk of developing a biofilm on or in a biologically relevant surface, or already has developed such a biofilm. Such a subject at risk can be a candidate for treatment with a D-amino acid in order to inhibit the development or onset of a biofilm-production-related disorder/condition or prevent the recurrence, onset, or development of one or more symptoms of a biofilm-related disorder or condition. Such a subject can be harboring an immature biofilm that is clinically evident or detectable to the skilled artisan, but that has not yet fully formed. A subject at risk of developing a biofilm can also be one in which implantation of an indwelling device, such as a medical device, is scheduled. The risk of developing a biofilm can also be due to a propensity of developing a biofilm-related disease (such as the presence of a channel transporter mutation associated with cystic fibrosis). In such subjects, a biofilm-related disorder can be at an early stage, e.g., no bacterial infection and/or biofilm formation is yet detected.

In a specific example, the methods described herein can be used to prevent biofilm formation in the airways of a cystic fibrosis patient. Such a patient can be treated while free of bacterial infection of the airways or upon detection of a bacterial infection.

The invention is further described in the following example, which does not limit the scope of the invention described in the claims. Room temperature denotes a temperature from the range of 20-25° C.

### EXAMPLES

### Materials and Methods

Strains and media. Bacillus subtilis NCIB3610 and its derivatives were grown in Luria-Bertani (LB) medium at 37° C. or MSgg medium (Branda et al., Proc. Natl. Acad. Sci. USA 98:11621 (2001)) at 23° C. Solid media contained 1.5% Bacto agar. When appropriate, antibiotics were added at the following concentrations for growth of B. subtilis: 10 μg per ml of tetracycline, and 5 μg per ml of erythromycin, 500 μg per ml of spectinomycin.

Strains used in this work:

All B. subtilis strains are derivatives of NCIB 3610, a wild strain that forms robust biofilms (Branda et al., Proc. Natl. Acad. Sci. USA 98:11621 (2001));

Strain FC5 (P_{epsA}-lacZ cat) (Chu et al., Mol. Microbiol. 59:1216 (2006));

Strain FC122 (P_{yqxM}-lacZ spec) (Chu et al., Mol. Microbiol. 59:1216 (2006));

Strain IKG55 (ΔracX::spec ΔylmE::tetR);

Strain DR-30 (tasA-mCherry cat);

Strain IKG40 (yqxM2);

Strain IKG44 (yqxM6);

Strain IKG50 (yqxM2 tasA-mCherry);

Strain IKG51 (yqxM6 tasA-mCherry);

Staphylococcus aureus SCOL from the Kolter lab collection.

Strain Construction.

Strains were constructed using standard methods (J. Sambrook, D. W. Russell, Molecular Cloning. A Laboratory Manual. (Cold Spring Harbor Laboratory Press, Cold Spring Harbor, N.Y., USA, 2001). Long-flanking PCR mutagenesis was used to create ΔracX::spec and ΔylmE::tetR (Wach, Yeast 12:259 (1996)). DNA was introduced into strain PY79 derivatives by DNA-mediated transformation of competent cells (Gryczan et al., J. Bacteriol. 134:318 (1978)). SPP1 phage-mediated transduction was used to introduce antibiotic resistance-linked mutations from PY79 derivatives into NCIB3610 (Yasbin et al., J. Virol. 14:1343 (1974)).

Reagents. Amino acids were obtained from Sigma-Aldrich (St. Louis, Mo.). ¹⁴C-D-tyrosine and ¹⁴C-L-proline were obtained from American Radiolabeled Chemicals, Inc (St. Louis, Mo.).

Colony and Pellicle Formation.

For colony formation on solid medium, cells were first grown to exponential growth phase in LB broth and 3 μl of culture were spotted onto solid MSgg medium containing 1.5% Bacto agar. The plates were incubated at 23° C. For pellicle formation in liquid medium, cells were grown to exponential phase and 6 μl of culture were mixed with 6 ml of medium in a 12-well plate (VWR). Plates were incubated at 23° C. Images of colonies and pellicles were taken using a SPOT camera (Diagnostic Instruments, USA).

Preparing Conditioned Medium.

Cells were grown in LB medium to exponential phase. 0.1 ml of culture was added to 100 ml of MSgg medium and grown without shaking in a 500 ml beaker at 23° C. Next, pellicles and conditioned medium was collected by centrifugation at 8,000 rpm for 15 min. The conditioned medium (supernatant fluid) was removed and filtered through a 0.22 μm filter. The filtrates were stored at 4° C. For further purification the biofilm-inhibiting activity was fractionated on a C-18 Sep Pak cartridge using stepwise elution of 0% to 100% methanol with steps of 5%.

Identification and Quantification of D-Amino Acids in the Conditioned Medium.

(A) Amino acid quantification. Standard solutions of Tyr, Leu, Met, and Trp were prepared at various concentrations (0.001-0.2 mM). These solutions were analyzed by LC/MS with a step gradient solvent system from 0% to 60% then to 100% CH₃CN with 0.1% formic acid (0-12-20 min) (Thermo Scientific Hypercarb 4.6 mm×100 mm, 5 μm) to obtain calibration curves of each amino acid concentration by ion count integration. Conditioned media samples were analyzed by LC/MS in the same manner to measure the total concentrations of all four chiral amino acids. (B) Identification of D-amino acids. The sample was dried in SpeedVac and dissolved in 100 μL 1 N NaHCO₃. 10 mg/mL of L-FDAA (N-(2,4-dinitro-5-fluoro-phenyl)-L-alanineamide) solution was prepared in acetone and 50 μL of the acetone solution was added to the sample in 1N NaHCO₃. The reaction mixture was incubated at 80° C. for 5 min and 50 μL of 2N HCl was added to quench the reaction. The derivatives were analyzed by LC/MS using a gradient solvent system from 10% to 100% CH₃CN with 0.1% formic acid over 30 min (Agilent 1200 Series HPLC/6130 Series MS, Phenomenex Luna C18, 4.6 mm×100 mm, 5 μm). The retention times of L-FDAA-amino acids were compared with L-FDAA-authentic standard amino acids.

Crystal Violet Staining.

Crystal violet staining was done as described previously (O'Toole et al., Mol. Microbiol. 30:295 (1998)) except that the cells were grown in 6-well plates. Wells were stained with 500 μl of 1.0% Crystal-violet dye, rinsed twice with 2 ml double-distilled water and thoroughly dried.

Fluorescence Microscopy.

For fluorescence microscopy analysis, 1 ml of culture was harvested. The cells were washed with PBS buffer and suspended in 50 μl of PBS buffer. Cover slides were pretreated with poly L-lysine (Sigma). Samples were examined using an Olympus workstation BX61 microscope. Images were taken using the automated software program SimplePCI and analyzed with program MetaMorph (Universal Imaging Corporation).

Transmission electron microscopy and immunolabeling. Samples were diluted with distilled water and adsorbed onto a carbon or formvar/carbon coated grid. The grid surface was made hydrophilic prior to use with glow discharge in a vacuum evaporator. Once the specimen was adsorbed onto the film surface, the excess sample was blotted off on a filter paper (Whatman #1) and the grid was floated on 5 μl of stain solution (1-2% aqueous uranyl acetate) for a few minutes and then blotted off. The samples were dried and examined in a Tecnai™ G² Spirit BioTWIN microscope at an accelerating voltage of 80 KV. Images were taken with an AMT 2k CCD camera.

For immunolocalization of TasA, diluted samples on nickel grids were floated on blocking buffer consisting of 1% nonfat dry milk in PBS with 0.1% Tween 20 for 30 min, incubated for 2 h with anti-TasA primary antibody diluted 1:150 in blocking buffer, rinsed in PBST, then exposed to goat-anti-rabbit 20 nm gold secondary antibody (Ted Pella, Inc., Redding, Calif.) for 1 h and rinsed. All grids were stained with uranyl acetate and lead citrate, then viewed as described above.

Assays of β-galactosidase activity. Cells were cultured in MSgg medium at 37° C. in a water bath with shaking 1 ml of culture was collected at each time point. β-galactosidase activity was determined as described previously (Chai et al., Mol. Microbiol. 67:254 (2008)).

Incorporation of amino acids into the cell wall. Cells in 50 ml of culture at the mid exponential phase of growth were harvested by centrifugation and washed with 0.05 M of phosphate buffer (pH 7) and re-suspended in 5 ml of the same buffer. Cells were either treated with 10 μCi/ml of ¹⁴C-D-tyrosine or ¹⁴C-L-proline and further incubated at 37° C. for 2 hours. The radioactivity of whole cells and cell wall fraction was monitored, and, at intervals samples were removed. For measurement of incorporation into whole cells, 0.1 ml samples were collected. For measurements of incorporation into cell wall, 0.5 ml samples were collected. The cells were harvested by centrifugation and re-suspended in SM buffer [0.5 M sucrose, 20 mM MgCl₂, and 10 mM potassium phosphate at pH (6.8)] containing 0.1 mg/ml lysozyme. The cells were then incubated at 37° C. for 10 min. Next, the resulting protoplasts were removed by centrifugation at 5000 rpm for 10 min, leaving the cell wall material in the supernatant fluid. That the cell wall fraction was free of protein was confirmed by immunoblot analysis using an anti-sigma A antibodies. Finally, 10 ml of 5% trichloroacetic acid was added to the whole cell samples and the cell wall material and maintained on ice for at least 30 min. The TCA-insoluble material was collected on Millipore filters (0.22 μm pore size, Millipore) and washed with 5% TCA. The filters were air-dried and placed in scintillation vials and the TCA-insoluble counts per minute were determined using a scintillation counter.

### Example 1

### Screening of D-Amino Acids in Biofilm Formation by B. Subtilis

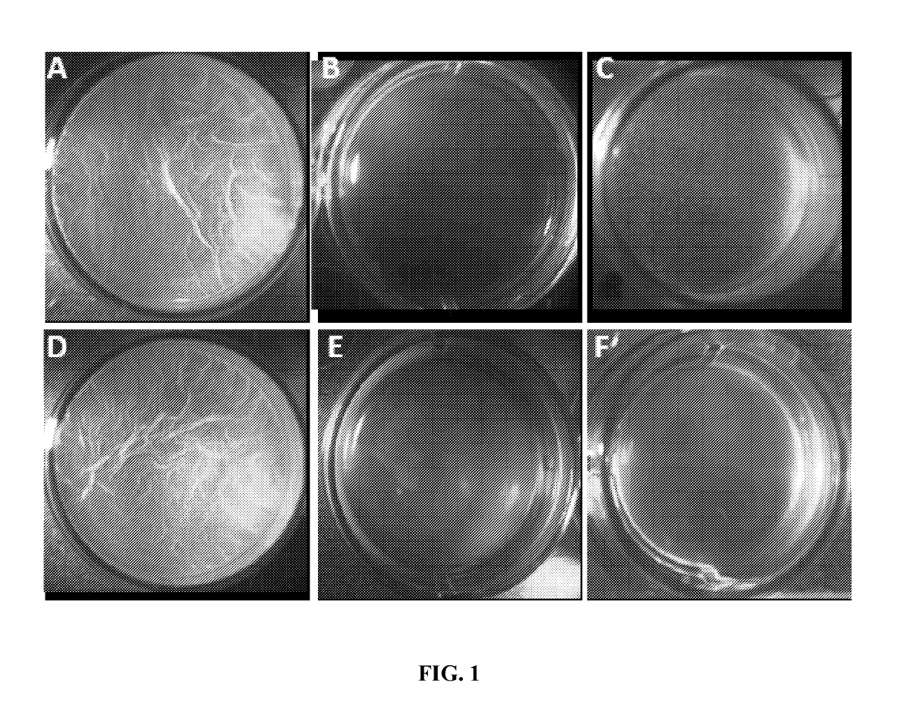

B. subtilis forms thick pellicles at the air/liquid interface of standing cultures after three days of incubation in biofilm-inducing medium (FIG. 1A). Upon incubation for an additional three to five days, however, the pellicle loses its structural integrity (FIG. 1-B). To investigate whether mature biofilms produce a factor that triggers biofilm disassembly, the effect of concentrated and partially purified extracts of conditioned medium on pellicle formation when added to fresh medium was assayed. To this end, conditioned medium from an eight-day-old culture was applied to a C18 Sep Pak column. Concentrated eluate from the column was then added to a freshly inoculated culture. An amount of concentrated eluate corresponding to 25% of the material from an equivalent volume of conditioned medium was sufficient to prevent pellicle formation (FIG. 1C). As a control, it was observed that addition of concentrated eluate prepared using conditioned medium from a three-day-old culture had little or no effect on pellicle formation (FIG. 1D). Further purification of the factor was achieved by eluting the cartridge in step-wise fashion with increasing concentrations of methanol. Elution with 40% methanol resulted in a fraction that was highly active in inhibiting pellicle formation (FIG. 1E). Yet, this material had little or no effect on cell growth. The biofilm-inhibiting activity was resistant to heating at 100° C. for 2 hours and proteinase K treatment (FIG. 1F).

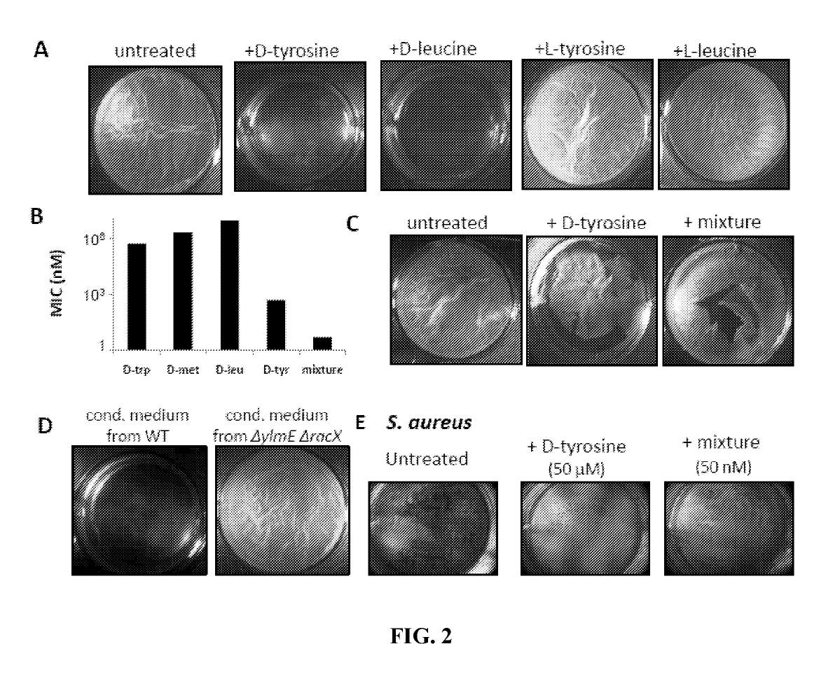

D-tyrosine, D-leucine, D-tryptophan, and D-methionine were screened for inhibiting biofilm formation by B. subtilis both in liquid and on solid medium (FIG. 2A, 5, 6). FIG. 2A shows the effects on pellicle formation of adding D-tyrosine (3 μM), D-leucine (8.5 mM), L-tyrosine (7 mM), or L-leucine (8.5 mM) to freshly inoculated cultures in biofilm-inducing medium after incubation for 3 days. Both D-tyrosine and D-leucine showed significant inhibition of biofilm growth, as compared to the corresponding L-amino acids. Similarly, FIG. 5 shows wells containing MSgg medium supplemented with D-tryptophan (0.5 mM), D-methionine (2 mM), L-tryptophan (5 mM) or L-methionine (5 mM) that were inoculated with strain NCIB3610 and incubated for 3 days. Only the D-amino acids were active in inhibiting biofilm formation.

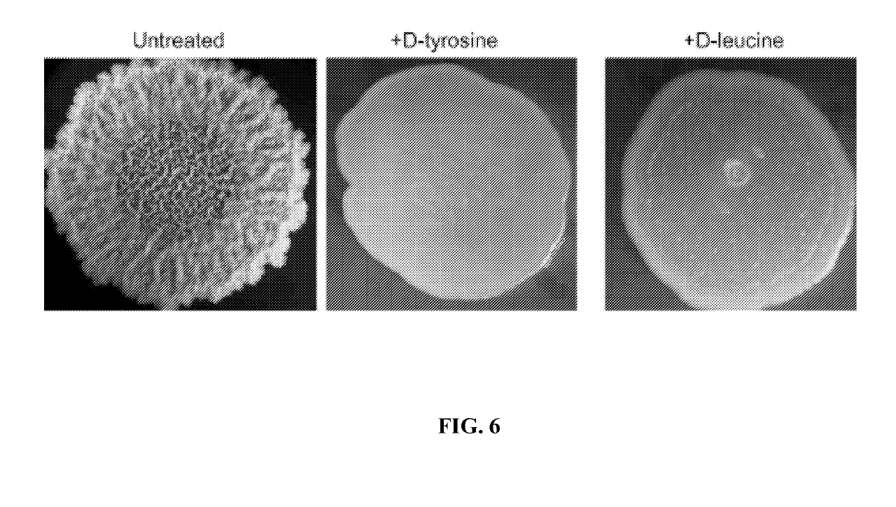

FIG. 6 shows plates containing solid MSgg medium supplemented with D-tyrosine (3 μM) or D-leucine (8.5 mM) that were inoculated with strain NCIB3610 and incubated for 4 days. Both D-tyrosine and D-leucine inhibited biofilm formation.

D-methionine, D-tryptophan, D-tyrosine and D-leucine showed significant inhibition of biofilm growth, as compared to the corresponding L-amino acids. In contrast, the corresponding L-isomers and D-isomers of other amino acids, such as D-alanine and D-phenylalanine, were not effective in the biofilm-inhibition assay for B. subtilis.

Next, the minimum concentration (MIC for Minimal Inhibitory Concentration) needed to prevent biofilm formation was determined. As shown in FIG. 2B, individual D-amino acids varied in their activity, with D-tyrosine being the most effective. D-methionine, D-tryptophan, and D-leucine had MICs of around 1 mM, while D-tyrosine has an MIC of about 100 nM. Strikingly, a mixture of all four D-amino acids (in equimolar amounts) was particularly potent, with a MBIC of <10 nM. Thus, D-amino acids act synergistically. The D-amino acids not only prevented biofilm formation but also disrupted existing biofilms. FIG. 2C shows 3 day-old cultures to which had been added no amino acids (untreated), D-tyrosine (3 μM) or a mixture of D-tyrosine, D-tryptophan, D-methionine and D-leucine (2.5 nM each), followed by further incubation for 8 hours. Addition of D-tyrosine or a mixture of the four D-amino acids caused the conspicuous breakdown of pellicles within a period of 8 hours.

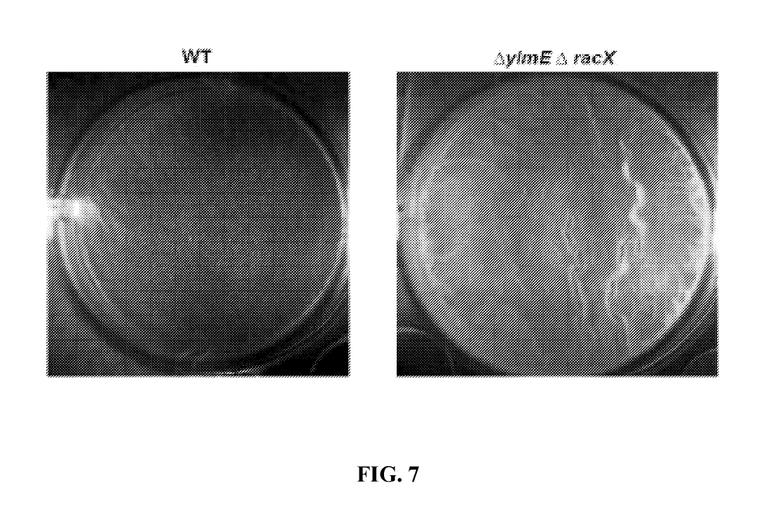

D-amino acids are generated by amino acid racemases, enzymes that convert the α-carbon stereocenter of these amino acids from L- to D-forms (Yoshimura et al., J. Biosci. Bioeng. 96:103 (2003)). Genetic evidence consistent with the idea that the biofilm-inhibiting factor is D-amino acids was obtained using mutants of ylmE and racX, genes whose predicted products exhibit sequence similarity to known racemases. Strains mutant for ylmE or racX alone showed a modest delay in pellicle disassembly (data not shown). FIG. 7 shows NCIB3610 (WT) and a mutant strain doubly deleted for ylmE and racX (IKG155) that were grown in 12 well plates and incubated for 5 days. Pellicles formed by cells doubly mutant for the putative racemases were significantly delayed in disassembly, suggesting that the strains in which racemase activity is especially reduced also exhibit reduced antibiofilm inhibition. Also, conditioned medium from the double mutant was ineffective in inhibiting biofilm formation, in contrast to conditioned medium from the wild type. FIG. 2D shows the effect of concentrated Sep Pak C-18 column eluate from conditioned medium from an 8-day-old culture from the wild type or from a strain (IKG55) doubly mutant for ylmE and racX, in which the double mutant shows significant biofilm buildup.

Next, it was determined whether D-amino acids were produced during biofilm maturation and in sufficient abundance to account for disassembly of mature biofilms. Accordingly, LC/MS was carried out, followed by the identification of the D-amino acids using derivatization with Nα-(2,4-dinitro-5-fluorophenyl)-L-alaninamide (L-FDAA) on conditioned medium collected at early and late times after pellicle formation. The results showed that D-tyrosine (6 μM), D-leucine (23 μM), and D-methionine (5 μM) were present at concentrations at or above those needed to inhibit biofilm formation by day 6 but at concentrations of <10 nM at day 3, e.g., at a level that is not sufficient to inhibit biofilm formation.

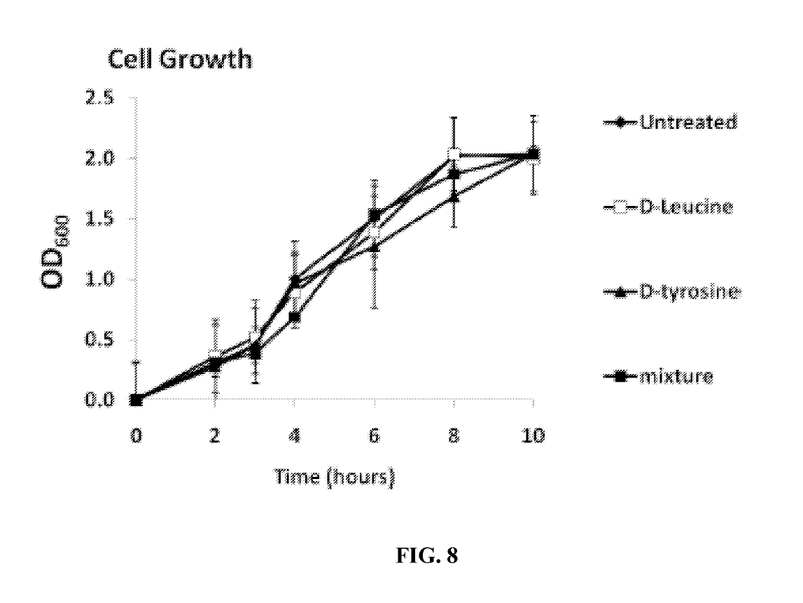

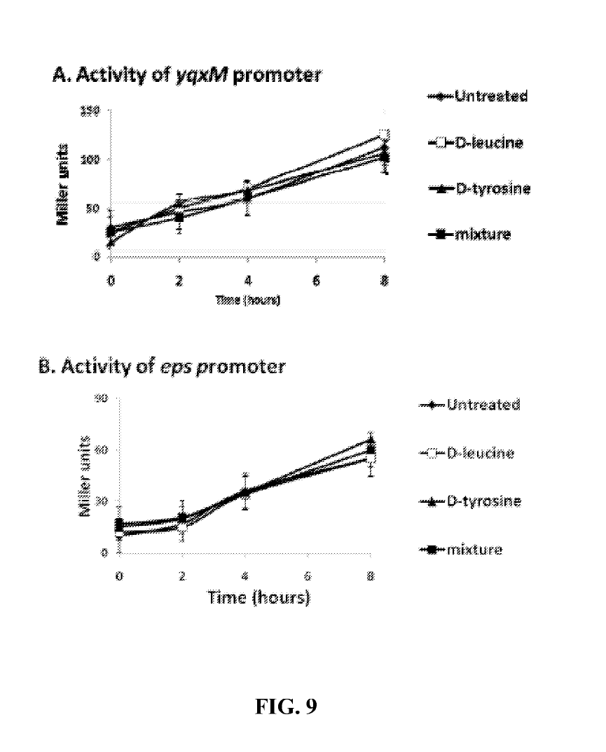

Similarly to the conditioned medium, D-amino acids did not inhibit cell growth, nor did they inhibit the expression of the matrix operons eps and yqxM (FIGS. 8-9). FIG. 8 shows the effect of D-amino acids on cell growth. Cells were grown in MSgg medium containing D-tyrosine (3 μM), D-leucine (8.5 mM) or the four D-amino acids mixture (2.5 nM each) with shaking Cell growth in the D-amino acid treated cultures was substantially the same as the untreated sample. FIG. 9A shows the expression of P_{yqxM}-lacZ by strain FC122 (carrying P_{yqxM}-lacZ) and FIG. 9B shows the expression of P_{epsA}-lacZ by strain FC5 (carrying P_{epsA}-lacZ) that were grown in MSgg medium containing D-tyrosine (3 μM), D-leucine (8.5 mM) or the four D-amino acids mixture (2.5 nM each) with shaking Again, yqxM and eps expression for the D-amino acid treated samples were substantially the same as the untreated sample.

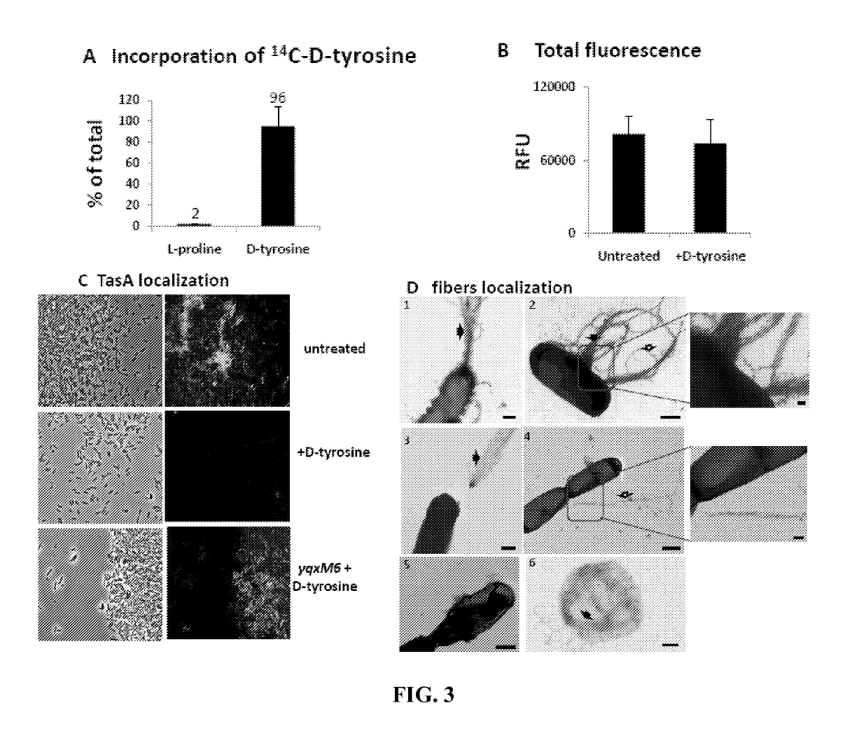

It was previously reported that D-amino acids are incorporated into the peptide cross bridge of the peptidoglycan component of the cell wall. To confirm, cells were grown in biofilm-inducing medium and incubated with either ¹⁴C-D-tyrosine or ¹⁴C-L-proline (10 μCi/ml) for 2 h at 37° C. FIG. 3A shows incorporation of radioactive D-tyrosine into the cell wall. Using ¹⁴C-D-tyrosine, D-tyrosine (but not ¹⁴C-L-proline) was shown to be incorporated into the cell wall. Results are presented as a percent of total incorporation into cells (360,000 cpm/ml for L-proline and 46,000 cpm/ml for D-tyrosine).

To investigate whether D-amino acids incorporated into the cell wall can disengage TasA fibers from being anchored to the cell, the localization of a functional fusion of TasA with the fluorescent reporter mCherry was examined. FIG. 3B shows total fluorescence from cells containing a functional tasA-mCherry translational fusion. The cells were grown to stationary phase with shaking in biofilm-inducing medium in the presence or absence of D-tyrosine (6 μM). As shown in FIG. 3B, treatment with D-tyrosine had little or no effect on the total accumulation of TasA-mCherry. When the cells were washed by centrifugation, resuspended and then examined by fluorescence microscopy, untreated cells (which were often in clumps) were seen to be intensely decorated with TasA-mCherry. In contrast, D-tyrosine-treated cells (which were largely unclumped) showed only low levels of fluorescence Similar results were obtained with D-leucine and with the four D-amino acid equimolar mixture. The localization of unmodified TasA protein was also analyzed by transmission electron microscopy using gold-labeled anti-TasA antibodies. FIG. 3D shows cell association of TasA fibers by electron microscopy. 24-hour-old cultures were incubated without (images 1 and 2) or with (images 3-6) D-tyrosine (0.1 mM) for an additional 12 hours. TasA fibers were stained by immunogold labeling using anti-TasA antibodies, and visualized by transmission electron microscopy as described in the Examples. The cells were mutant for the eps operon (Δeps) as the absence of exopolysaccharide significantly improves the imaging of TasA fibers. Filled arrows indicate fiber bundles; open arrows indicate individual fibers. The scale bar is 500 nm. The scale bar in the enlargements of images 2, 4 and 6 is 100 nm. Images 1 and 2 show fiber bundles attached to cells, images 3, 4 and 6 show individual fibers and bundles detached from cells, and images 3-5 show cells with little or no fiber material. TasA fibers were seen as being anchored to the cells of untreated pellicles (FIG. 3D, images 1 and 2). In contrast, cells treated for 12 hours with D-tyrosine consisted of a mixture of cells that were largely undecorated with TasA fibers and free TasA fibers or aggregates of fibers that were not anchored to cells (FIG. 3D, images 3-6). Without wishing to be bound by theory, one of the mechanisms by which D-tyrosine treats biofilms may be to induce the shedding of fibers by the cells.

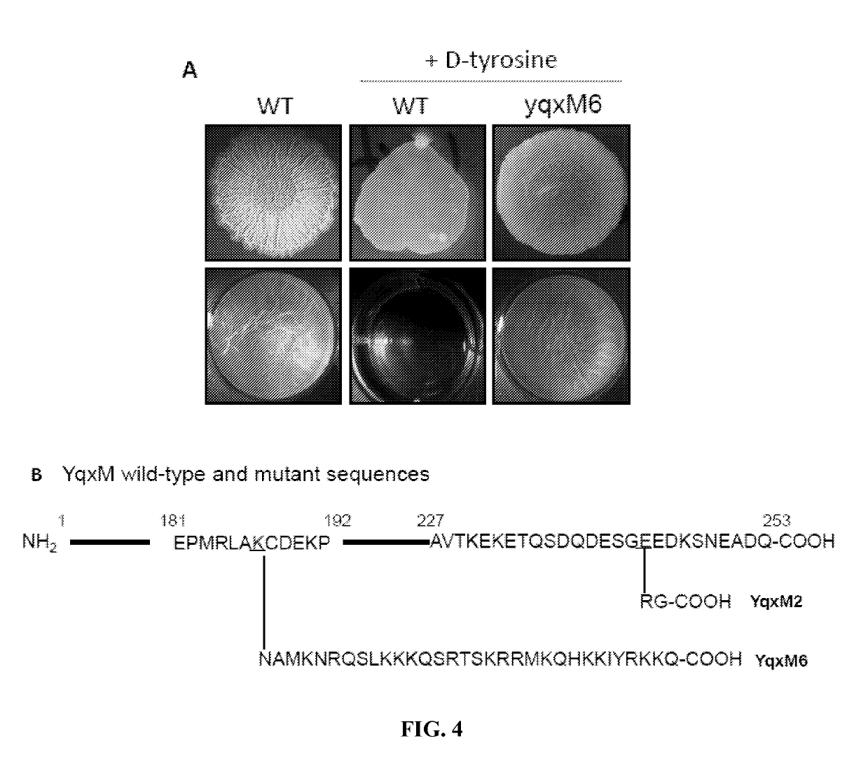

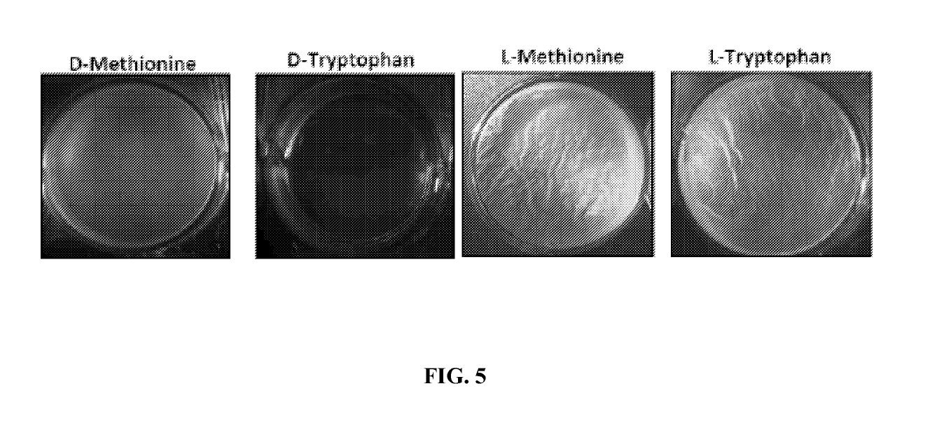

Genetic evidence that D-amino acids act by disrupting the anchoring of TasA fibers to the cells was obtained from the isolation of D-tyrosine resistant mutants. FIG. 4A shows cells grown for 3 days on solid (top images) or liquid (bottom images) biofilm-inducing medium that did or did not contain D-tyrosine. Wrinkled papillae appeared spontaneously on the flat colonies formed during growth on solid medium containing D-tyrosine (FIG. 4A) or D-leucine (data not shown). Importantly, no such papillae appeared on plates containing all four active D-amino acids. When purified, these spontaneous mutants gave rise to wrinkled colonies and pellicles in the presence of D-tyrosine or D-leucine. Several such mutants were isolated and most of them contained a mutation in or near the yqxM operon. Two mutations were examined in detail and found to be frame-shift mutations near the 3′ end of the 759 base-pair-long yqxM gene. yqxM2 was an insertion of G:C at base pair 728 in the yqxM open-reading frame and yqxM6 was a deletion of A:T at base pair 568 (FIG. 4B). FIG. 4B shows an abbreviated amino acid sequence for YqxM. Underlined are residues specified by codons in which the yqxM2 and yqxM6 frame-shift mutations resulted in the indicated sequence changes.

FIG. 3C shows cell association of TasA-mCherry by fluorescence microscopy. Wild-type cells and yqxM6 (IKG51) mutant cells containing the tasA-mCherry fusion were grown to stationary phase (OD=1.5) with shaking in biofilm-inducing medium in the presence or absence (untreated) of D-tyrosine (6 μM) as indicated, washed in PBS, and visualized by fluorescence microscopy. Fluorescence microscopy showed that the presence of yqxM2 and yqxM6 restored clumping and cell decoration by TasA-mCherry to cells treated with D-tyrosine (FIG. 3C). Previous work has shown that YqxM is required for the association of TasA with cells (Branda et al., Mol. Microbiol. 59:1229 (2006)). Without wishing to be bound by theory, this discovery that the biofilm-inhibiting effect of D-amino acids can be overcome by mutants of YqxM reinforces the view that the effect of D-amino acid incorporation into the cell wall is to impair the anchoring of the TasA fibers to the cell. A domain near the C-terminus of YqxM may trigger the release of TasA in response to the presence of D-tyrosine or D-leucine in the cell wall.

### Example 2

### Screening of D-Amino Acids in Biofilm Formation by S. aureus and P. aeruginosa

The effect of D-amino acids on biofilm formation by other bacteria was examined. The pathogenic bacterium Staphylococcus aureus forms biofilms on plastic surfaces (Otto, Curr. Top. Microbiol. Immunol. 322:207 (2008)), which can be detected by washing away unbound cells and staining the bound cells with crystal violet. FIG. 2E shows S. aureus (strain SCO1) that had been grown in 12-well polystyrene plates for 24 hours at 37° C. in TSB medium containing glucose (0.5%) and NaCl (3%). Additionally added to the wells were no amino acids (untreated), D-tyrosine (50 μM) or the D-amino acid mixture (15 nM each). Cells bound to the polystyrene were visualized by washing away unbound cells and then staining with crystal violet. FIG. 2E shows that 50 μM concentrations of D-tyrosine and 50 nM concentrations of mixed D-amino acids (D-tyrosine, D-leucine, D-tryptophan, and D-methionine; 50 nM each) were highly effective in preventing biofilm formation by the pathogenic bacterium.

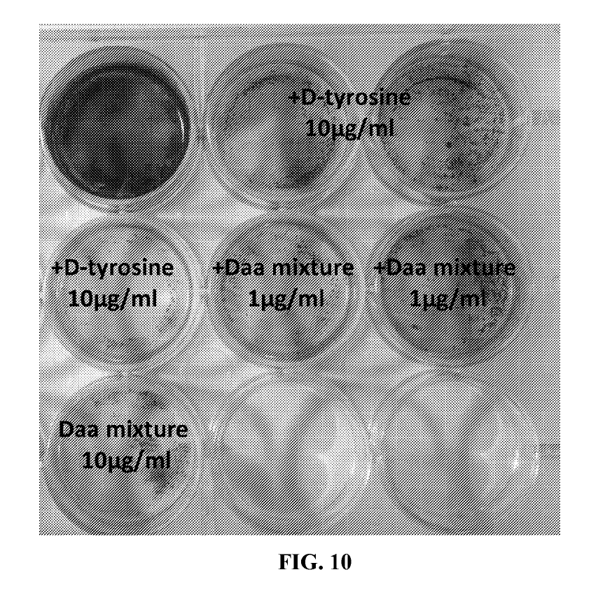

In addition, FIG. 10 demonstrates that 10 μM of D-tyrosine was effective in preventing biofilm formation by Pseudomonas aeruginosa, whereas 1 μM of an equimolar mix of D-tyrosine, D-leucine, D-tryptophan, and D-methionine was effective. FIG. 10 shows the inhibition of Pseudomonas aeruginosa biofilm formation by D-amino acids. P. aeruginosa strain P014 was grown in 12-well polystyrene plates for 48 hours at 30° C. in M63 medium containing glycerol (0.2%) and Casamino acids (20 m/ml). Additionally added to the wells were no amino acids (untreated), D-tyrosine or the D-amino acid equimolar mixture. Cells bound to the polystyrene were visualized by washing away unbound cells and then staining with crystal violet. Wells were stained with 500 μA of 1.0% Crystal-violet dye, rinsed twice with 2 ml double-distilled water and thoroughly dried.

### Example 3

### D-Amino Acids Mixtures Active in Inhibiting Staphylococcus Aureus and Pseudomonas aeruginosa Biofilms

Two different mixtures are very active in preventing the formation of Staphylococcus aureus biofilms. One is an equimolar mixture of D-tyrosine, D-methionine, D-leucine and D-tryptophan. The D-aa mixture of D-trp, D-met, D-tyr and D-leu was active in significantly lower concentration than the individual amino acids in all tested bacterial strains B. subtilis, Staphylococcus aureus (FIG. 11), and Pseudomonas aeruginosa (FIG. 12). For experiments reported in Table 1, the organism/strain was S.a. Harvard SCO1, the culture medium was TSB and the cell inoculation was at 2×10⁹ cfu. For experiments reported in Table 2, the organism/strain was S.a. Harvard PA14, the culture medium was M63 and the cell inoculation was at 1.5×10⁹ cfu. Biofilm was visualized using the crystal violet method. The data is shown below in Tables 1 and 2:

TABLE 1
(Data for FIG. 11)

| | | | | % Inhibition |
|---|---|---|---|---|
| | | | | relative to |
| | | | | control |
| | Incubation | | | (0%, <50%, |
| Example | Time/ | Active/ | | 50-90%, |
| No. | Temperature | Concentration | Substrate | >90%) |
| Untreated | 24 h/37° C. | 0/0 | Polystyrene | 0 |
| (1 row) | | | | |
| 11.1 | 24 h/37° C. | D-Tyr/100 nM | Polystyrene | 0 |
| 11.2 | 24 h/37° C. | D-Tyr/10 μM | Polystyrene | 0 |
| 11.3 | 24 h/37° C. | D-Tyr/100 μM | Polystyrene | >90% |
| 11.4 | 24 h/37° C. | D-Tyr/500 μM | Polystyrene | >90% |
| 11.5 | 24 h/37° C. | D-Met/100 nM | Polystyrene | 0 |
| 11.6 | 24 h/37° C. | D-Met/10 μM | Polystyrene | 0 |
| 11.7 | 24 h/37° C. | D-Met/100 μM | Polystyrene | 0 |
| 11.8 | 24 h/37° C. | D-Met/500 μM | Polystyrene | 0 |
| 11.9 | 24 h/37° C. | D-Leu/100 nM | Polystyrene | 0 |
| 11.10 | 24 h/37° C. | D-Leu/10 μM | Polystyrene | 0 |
| 11.11 | 24 h/37° C. | D-Leu/100 μM | Polystyrene | 0 |
| 11.12 | 24 h/37° C. | D-Leu/500 μM | Polystyrene | 0 |
| 11.13 | 24 h/37° C. | D-Trp/100 nM | Polystyrene | 0 |
| 11.14 | 24 h/37° C. | D-Trp/10 μM | Polystyrene | 0 |
| 11.15 | 24 h/37° C. | D-Trp/100 μM | Polystyrene | <50% |
| 11.16 | 24 h/37° C. | D-Trp/500 μM | Polystyrene | <50% |
| 11.17 | 24 h/37° C. | D-Met/D-Leu/D- | Polystyrene | >90% |
| | | Trp/D-Tyr mix/ | | |
| | | 100 nM | | |
| 11.18 | 24 h/37° C. | D-Met/D-Leu/D- | Polystyrene | >90% |
| | | Trp/D-Tyr mix/ | | |
| | | 10 μM | | |

TABLE 2
(Data for FIG. 12)

| | | | | % Inhibition |
|---|---|---|---|---|
| | | | | relative to |
| | | | | control |
| | Incubation | | | (0%, <50%, |
| Example | Time/ | Active/ | | 50-90%, |
| No. | Temperature | Concentration | Substrate | >90%) |
| Untreated | 48 h/30° C. | 0/0 | Polystyrene | 0 |
| (1 row) | | | | |
| 12.1 | 48 h/30° C. | D-Trp/100 nM | Polystyrene | 0 |
| 12.2 | 48 h/30° C. | D-Trp/10 μM | Polystyrene | <50% |
| 12.3 | 48 h/30° C. | D-Trp/100 μM | Polystyrene | 50-90% |
| 12.4 | 48 h/30° C. | D-Trp/500 μM | Polystyrene | 50-90% |
| 12.5 | 48 h/30° C. | D-Met/100 nM | Polystyrene | 0 |
| 12.6 | 48 h/30° C. | D-Met/10 μM | Polystyrene | 0 |
| 12.7 | 48 h/30° C. | D-Met/100 μM | Polystyrene | 0 |
| 12.8 | 48 h/30° C. | D-Met/500 μM | Polystyrene | 0 |
| 12.9 | 48 h/30° C. | D-Leu/100 nM | Polystyrene | 0 |
| 12.10 | 48 h/30° C. | D-Leu/10 μM | Polystyrene | 0 |
| 12.11 | 48 h/30° C. | D-Leu/100 μM | Polystyrene | 0 |
| 12.12 | 48 h/30° C. | D-Leu/500 μM | Polystyrene | 0 |
| 12.13 | 48 h/30° C. | D-Tyr/100 nM | Polystyrene | 0 |
| 12.14 | 48 h/30° C. | D-Tyr/10 μM | Polystyrene | >90% |
| 12.15 | 48 h/30° C. | D-Tyr/100 μM | Polystyrene | >90% |
| 12.16 | 48 h/30° C. | D-Tyr/500 μM | Polystyrene | >90% |
| 12.17 | 48 h/30° C. | D-Met/D-Leu/D- | Polystyrene | >90% |
| | | Trp/D-Tyr mix/ | | |
| | | 100 nM | | |
| 12.18 | 48 h/30° C. | D-Met/D-Leu/D- | Polystyrene | >90% |
| | | Trp/D-Tyr mix/ | | |
| | | 10 μM | | |

The equimolar mixture of D-tyrosine, D-phenylalanine, D-proline is even more effective than the above mixture. Also, the mixture was more active as a mixture than each of the amino acids individually (FIGS. 13 and 14). For experiments reported in Tables 3 and 4, the organism/strain was S.a. Harvard SCO1, the culture medium was TSB and the cell inoculation was at 2×10⁹ cfu. Biofilm was visualized using the crystal violet method. The data is shown in Tables 3 and 4:

TABLE 3
(Data for FIG. 13)

| | | | | % Inhibition |
|---|---|---|---|---|
| | | | | relative to |
| | | | | control |
| | Incubation | | | (0%, <50%, |
| Example | Time/ | Active/ | | 50-90%, |
| No. | Temperature | Concentration | Substrate | >90%) |
| Untreated | 24 h/37° C. | 0/0 | Polystyrene | 0 |
| (1 row) | | | | |
| 13.1 | 24 h/37° C. | D-Phe/10 μM | Polystyrene | <50% |
| 13.2 | 24 h/37° C. | D-Phe/100 μM | Polystyrene | <50% |
| 13.3 | 24 h/37° C. | D-Phe/500 μM | Polystyrene | >90% |
| 13.4 | 24 h/37° C. | D-Pro/1 mM | Polystyrene | >90% |
| 13.5 | 24 h/37° C. | D-Pro/10 μM | Polystyrene | <50% |
| 13.6 | 24 h/37° C. | D-Pro/100 μM | Polystyrene | <50% |
| 13.7 | 24 h/37° C. | D-Pro/500 μM | Polystyrene | >90% |
| 13.8 | 24 h/37° C. | D-Pro/1 mM | Polystyrene | >90% |
| 13.9 | 24 h/37° C. | D-Pro/D-Phe/D- | Polystyrene | >90% |
| | | Tyr mix/100 nM | | |
| 13.10 | 24 h/37° C. | D-Pro/D-Phe/D- | Polystyrene | >90% |
| | | Tyr mix/10 μM | | |

TABLE 4
(Data for FIG. 14)

| | | | | | % Inhibition |
|---|---|---|---|---|---|
| | | | | | relative to |
| | | | | | control |
| Exam- | | Incubation | Active/ | | (0%, <50%, |
| ple | Repli- | Time/ | Concen- | | 50-90%, |
| No. | cates | Temperature | tration | Substrate | >90%) |
| Medium | 4 | 24 h/37° C. | 0/0 | Polystyrene | 0 |
| control | | | | | |
| 14.1 | 4 | 24 h/37° C. | L-Met/L- | Polystyrene | 0% |
| | | | Leu/L- | | |
| | | | Trp/L- | | |
| | | | Tyr mix/ | | |
| | | | 1 mM | | |
| 14.2 | 4 | 24 h/37° C. | L-Pro/L- | Polystyrene | 0% |
| | | | Phe/L-Tyr | | |
| | | | mix/1 mM | | |

### Example 4

### Alternative Quantification Method for Biofilm Formation in Staphylococcus aureus

Planktonic cells were completely removed by a Gilson pipette, followed by tapping over a paper towel. Then a photographic image of the biofilm plates was taken carefully against black background (FIGS. 15 and 16). For experiments reported in Tables 5 and 6, the organism/strain was S.a. Harvard SCO1, the culture medium was TSB and the cell inoculation was at 2×10⁹ cfu. Biofilm was visualized using the visual against black background as the method. The data is shown in Tables 5 and 6:

TABLE 5
(Data for FIG. 15)

| | | | | | | % |
|---|---|---|---|---|---|---|
| | | | | | | Inhibition |
| | | | | | | relative to |
| | | | | | | control |
| | | Incubation | | | | (0%, <50%, |
| | | Time/ | Active/ | | Visualization | 50-90%, |
| Example No. | Replicates | Temperature | Concentration | Substrate | Method | >90%) |
| Untreated | 3 | 24 h/37° C. | 0/0 | Polystyrene | Visual | 0 |
| | | | | | against | |
| | | | | | black | |
| | | | | | background | |
| 15.1 | 3 | 24 h/37° C. | D-Pro/D- | Polystyrene | Visual | >90% |
| | | | Phe/D-Tyr | | against | |
| | | | mix/10 μM | | black | |
| | | | | | background | |
| 15.2 | 3 | 24 h/37° C. | D-Pro/D- | Polystyrene | Visual | >90% |
| | | | Phe/D-Tyr | | against | |
| | | | mix/100 μM | | black | |
| | | | | | background | |
| 15.3 | 3 | 24 h/37° C. | D-Pro/D- | Polystyrene | Visual | >90% |
| | | | Phe/D-Tyr | | against | |
| | | | mix/500 μM | | black | |
| | | | | | background | |

TABLE 6
(Data for FIG. 16

| | | | | | | % Inhibition |
|---|---|---|---|---|---|---|
| | | | | | | relative to |
| | | | | | | control |
| | | Incubation | | | | (0%, <50%, |
| Example | | Time/ | Active/ | | Visualization | 50-90%, |
| No. | Replicates | Temperature | Concentration | Substrate | Method | >90%) |
| Untreated | 3 | 24 h/37° C. | 0/0 | Polystyrene | Visual | 0 |
| | | | | | against | |
| | | | | | black | |
| | | | | | background | |
| 16.1 | 3 | 24 h/37° C. | L-Pro/L- | Polystyrene | Visual | 0 |
| | | | Phe/L-Tyr | | against | |
| | | | mix/10 μM | | black | |
| | | | | | background | |
| 16.2 | 3 | 24 h/37° C. | L-Pro/L- | Polystyrene | Visual | 0 |
| | | | Phe/L-Tyr | | against | |
| | | | mix/ | | black | |
| | | | 100 μM | | background | |
| 16.3 | 3 | 24 h/37° C. | L-Pro/L- | Polystyrene | Visual | 0 |
| | | | Phe/L-Tyr | | against | |
| | | | mix/ | | black | |
| | | | 500 μM | | background | |

Biofilm cells were removed from the above plates in Tables 5 and 6 by re-suspension in PBS, and their OD600 was determined using spectrophotometer (FIG. 17). For experiments reported in Table 7, the organism/strain was S.a. Harvard SCO1, the culture medium was TSB and the cell inoculation was at 2×10⁹ cfu. Biofilm was visualized by measuring OD600 of absorbed bacteria. The data is shown in Table 7:

TABLE 7
(Data for FIG. 17)

| | Incubation | | | | Measured |
|---|---|---|---|---|---|
| | Time/ | Active/ | | Visualization | Optical |
| Example No. | Temperature | Concentration | Substrate | Method | Density |
| Not Treated | 24 h/37° C. | 0/0 | Polystyrene | Measuring OD₆₀₀ of | 6.5 |
| (NT) | | | | absorbed bacteria | |
| 17.1 | 24 h/37° C. | D-Pro/D- | Polystyrene | Measuring OD₆₀₀ of | 1.5 |
| | | Phe/D-Tyr mix/ | | absorbed bacteria | |
| | | 10 μM | | | |
| 17.2 | 24 h/37° C. | D-Pro/D- | Polystyrene | Measuring OD₆₀₀ of | 0.8 |
| | | Phe/D-Tyr mix/ | | absorbed bacteria | |
| | | 100 μM | | | |
| 17.3 | 24 h/37° C. | D-Pro/D- | Polystyrene | Measuring OD₆₀₀ of | 0.7 |
| | | Phe/D-Tyr mix/ | | absorbed bacteria | |
| | | 500 μM | | | |
| 17.4 | 24 h/37° C. | L-Pro/L- | Polystyrene | Measuring OD₆₀₀ of | 6.4 |
| | | Phe/L-Tyr mix/ | | absorbed bacteria | |
| | | 10 μM | | | |
| 17.5 | 24 h/37° C. | L-Pro/L- | Polystyrene | Measuring OD₆₀₀ of | 6.5 |
| | | Phe/L-Tyr mix/ | | absorbed bacteria | |
| | | 100 μM | | | |
| 17.6 | 24 h/37° C. | L-Pro/L- | Polystyrene | Measuring OD₆₀₀ of | 6.5 |
| | | Phe/L-Tyr mix/ | | absorbed bacteria | |
| | | 500 μM | | | |

### Example 5

### Effect of D-Amino Acids on Staphylococcus aureus Biofilm Formation on Epoxy Surfaces

To test the possibility of developing controlled release methods of D-amino acids from different surfaces, epoxy surfaces were incubated for 24 hrs in D-amino acids mixtures. They were completely dried and incubated in a fresh TSB medium inoculated with Staphylococcus aureus. For experiments reported in Tables 8 and 9, the organism/strain was S.a. Harvard SCO1, the culture medium was TSB and the cell inoculation was at 2×10⁹ cfu. Biofilm was visualized using visual against black background. As shown in FIGS. 18 and 19, D-aa mixtures (as described above) dramatically decreased Staphylococcus aureus biofilm formation on the soaked substrates. The data is shown in Tables 8 and 9:

TABLE 8
(Data for FIG. 18)

| | | | | | % Inhibition |
|---|---|---|---|---|---|
| | | | | | relative to |
| | | | | | control |
| | Incubation | | | | (0%, <50%, |
| | Time/ | Active/ | | Visualization | 50-90%, |
| Example No. | Temperature | Concentration | Substrate | Method | >90%) |
| 18.1 | 24 h/37° C. | L-met/Leu/L- | Epoxy | Visual against | 0% |
| | | Trp/L-Tyr mix/ | | black | |
| | | 1 mM | | background | |
| 18.2 | 24 h/37° C. | D-Met/D- | Epoxy | Visual against | >90% |
| | | Leu/D-Trp/D- | | black | |
| | | Tyr mix/1 mM | | background | |

TABLE 9
(Data for FIG. 19)

| | | | | | % Inhibition |
|---|---|---|---|---|---|
| | | | | | relative to |
| | | | | | control |
| | Incubation | | | | (0%, <50%, |
| | Time/ | Active/ | | Visualization | 50-90%, |
| Example No. | Temperature | Concentration | Substrate | Method | >90%) |
| 19.1 | 24 h/37° C. | L-Pro/L- | Epoxy | ″ | 0% |
| | | Phe/L-Tyr | | | |
| | | mix/500 μM | | | |
| 19.2 | 24 h/37° C. | D-Pro/D- | Epoxy | Visual against | >90% |
| | | Phe/D-Tyr mix/ | | black | |
| | | 500 μM | | background | |

Additionally, Norland Optical Adhesive 61 surfaces were incubated with D-tyrosine, D-proline, D-phenylalanine for 24 hrs. They were completely dried and incubated in a fresh TSB medium inoculated with Staphylococcus aureus. The D-aa mixture (but not the L-mixture) dramatically decreased Staphylococcus aureus biofilm formation.

For this example, polymer substrates were molded in polydimethylsiloxane (SYLGARD 184, Dow Corning) from UVO-114 (Epoxy Technology) and Norland Optical Adhesive 61 (Norland Products) UV-curable polymers.

### Example 6

### Additional Ways to Observe D-Amino Acids Effect on Biofilm Formation in Pseudomonas aeruginosa

Similarly to Bacillus subtilis, Pseudomonas aeruginosa forms a complex architecture on defined medium. These complex structures require the proper formation and assembly of the extra-cellular matrix. Addition of D-tyrosine (500 μM) or D-tryptophan (500 μM) inhibited biofilm formation on defined medium in Pseudomonas aeruginosa (FIG. 20) while addition of L-tyrosine (500 μM) and L-tryptophan did not. Similar results were obtained with Bacillus subtilis. For these experiments, the organism/strain was P.a. Harvard PA14, the culture medium was M63 and the cell inoculation was at 1.5×10⁹ cfu.

An alternative method to observe biofilm formation on a 6 well plate with or without D-amino acids and using Syto-9 staining was as follows: Pseudomonas aeruginosa biofilms were washed twice with PBS and then fixed for at least an hour in 5% Glutaraldehyde in PBS. The fixed biofilms were then rinsed once again with PBS and soaked in 0.1% v/v Triton X-100 in PBS (PBST) for 15 minutes. The solution was exchanged with 0.1 nM SYTOX green (Invitrogen) in cold PBST and gently rocked in the dark for at least 15 minutes. Fluorescence images of the biofilms were captured with a Leica DMRX compound microscope using a Xe lamp and a K3 Leica filtercube. As shown in FIG. 21, there was a dramatic decrease in the number of cells attached to the bottom of the biofilm plate in the presence of D-tyrosine. The amount of attached single cells was quantified using image J. The decrease in the amount of cells attached to the epoxy surfaces soaked with D-aa compared with the L-aa control was substantially more.

TABLE 10
(Data for FIG. 21)

| | | | | | % Inhibition |
|---|---|---|---|---|---|
| | | | | | relative to |
| | | | | | control |
| | Incubation | | | | (0%, <50%, |
| | Time/ | Active/ | | Visualization | 50-90%, |
| Example No. | Temperature | Concentration | Substrate | Method | >90%) |
| Positive | 12 h/30° C. | 0 | Polystyrene | Syto-9 staining | 0 |
| control | | | | | |
| 21.1 | 12 h/30° C. | D-Tyr/50 μM | Polystyrene | Syto-9 staining | >90% |
| 21.2 | 12 h/30° C. | L-Tyr/500 μM | Polystyrene | Syto-9 staining | 0 |

### Example 7

### Assessing the Effect of D-Amino Acids on a Gram Negative Pathogens

To assess the possibility for a broad-spectrum anti biofilm activity the efficient equimolar quartet of D-tyrosine, D-phenylalanine, and D-proline was tested against the gram negative pathogen Proteus mirabilis. As shown in FIG. 22, the D-aa mixture was active against Proteus mirabilis. Biofilm in Table 11 was visualized using the crystal violet method. The data is shown in Tables 11:

TABLE 11
(Data for FIG. 22)

| | | | | | | | % Inhibition |
|---|---|---|---|---|---|---|---|
| | | | | | | | relative to |
| | | | | | | | control |
| | | | | Incubation | | | (0%, <50%, |
| Example | Organism/ | | Innoculation | Time/ | Active/ | | 50-90%, |
| No. | Strain | Medium | cfu | Temperature | Concentration | Substrate | >90%) |
| Positive | Proteus | LB | 2+E09 | 48 h/30° C. | 0 | Polystyrene | 0 |
| control | mirabilis. | | | | | | |
| | Harvard | | | | | | |
| 22.1 | Proteus | LB | 2+E09 | 48 h/30° C. | D-Met/D- | Polystyrene | >90% |
| | mirabilis.. | | | | Leu/D-Trp/D- | | |
| | Harvard | | | | Tyr mix/ | | |
| | | | | | 100 μM | | |
| 2 | Proteus | LB | 2+E09 | 48 h/30° C. | L-Met/L- | Polystyrene | 0 |
| | mirabilis. | | | | Leu/L-Trp/L- | | |
| | Harvard | | | | Tyr mix/ | | |
| | | | | | 100 μM | | |

### Example 8

### Assessing the Effect of D-Amino Acids on a Gram Positive Pathogen

To assess the possibility for a broad-spectrum anti biofilm activity the efficient equimolar quartet of D-tyrosine, D-phenylalanine, and D-proline was tested against the gram positive pathogen Streptococcus mutans. As shown in FIG. 23, the D-aa mixture was active against Streptococcus mutans. Biofilm in Table 12 was visualized using the crystal violet method. The data is shown in Tables 12:

TABLE 12
(Data for FIG. 23)

| | | | | | | | % Inhibition |
|---|---|---|---|---|---|---|---|
| | | | | | | | relative to |
| | | | | | | | control |
| | | | | Incubation | | | (0%, |
| Example | Organism/ | | Innoculation | Time/ | Active/ | | <50%, 50-90%, |
| No. | Strain | Medium | cfu | Temperature | Concentration | Substrate | >90%) |
| 23.1 | Streptococcus | BHI + | 2+E09 | 72 h/37° C. | L-Met/L- | Polystyrene | 0 |
| | mutans. | sucrose | | | Leu/L-Trp/L- | | |
| | Temple | | | | Tyr mix/1 mM | | |
| 23.2 | Streptococcus | BHI + | 2+E09 | 72 h/37° C. | D-Met/D- | Polystyrene | >90% |
| | mutans. | sucrose | | | Leu/D-Trp/D- | | |
| | Temple | | | | Tyr mix/1 mM | | |

### Examples Related to Coatings that can be Used in Medical Devices

### Example 9

### Coating Containing D-Tyrosine

D-Tyrosine, 0.5%, by weight based on the weight of the resin solids, is incorporated into a two-component polyester urethane coating based on a commercially available polyester polyol and commercially available isocyanurate. The coating system is catalyzed with 0.015% dibutyl tin dilaurate based on total resin solids.

The coating formulation is applied by drawdown onto transparent glass slides approximately 4″×6″ to a film thickness of about 2 mils (0.002″).

These films are cured in an oven at 120° F. (49° C.) oven.

### Example 10

### Polymer Containing D-Amino Acid Mixture

Liquid silicone rubber sheets are prepared as described in U.S. Pat. No. 5,973,030. Further included in the formulations are 0.01 to 1 weight percent D amino acid mixture, in a ratio 1:1:1:1 of D-Tryosine:D-Leucine:D-Methionine:D-Tryptophan.

### Example 11

### Water Based Coating Containing D-Amino Acid Mixture

Water based clear acrylic industrial coating formulation containing 1 weight percent D amino acid mixture, in a ratio 1:1:1:1 of D-Tyrosine:D-Leucine:D-Methionine:D-Tryptophan is coated onto glass slides at 2 mil thickness.

### Example 12

### Solvent Based Coating Containing D-Amino Acid Mixture

A solvent based polyurethane coating is prepared containing 1 weight percent D amino acid mixture, in a ratio 1:1:1:1 of D-Tyrosine:D-Leucine:D-Methionine:D-Tryptophan. The coating is applied to glass slides at 2 mil thickness.

### Example 13

### UV Curable Water Based Coating Containing D-Amino Acid Mixture

A clear UV curable water-borne industrial coating is formulated by mixing with high speed stirrer the ingredients (see table below).

| | Weight-% |
|---|---|
| Alberdingk Lux 399 | 97.8 |
| (acrylate polyurethane copolymer dispersion), Alberdingk | |
| Boley | |
| Borchigel L 75 N (thickener), Borchers | 0.3 |
| Byk 347 (wetting agent), Byk Chemie | 0.4 |
| IRGACURE 500 (photoinitiator), Ciba | 1.0 |
| D-amino acid mixture | 0.5 |

To the prepared formulation, D amino acid mixture, in a ratio 1:1:1:1 of D-Tryosine:D-Leucine:D-Methionine:D-Tryptophan.is added, and stirred at high shear rate (2000 rpm) for 30 minutes at room temperature. For the purpose of comparison, control formulations containing no D amino acids are prepared in the same manner.

The coating is applied with a 50 μm slit coater to white coated aluminum panels, dried 10 minutes at 60° C. and cured with two medium pressure mercury vapor lamps (2×80 W/cm) at 5 m/min.

### Example 14

### Solvent Based Coating Containing D-Amino Acid Mixture

2 Pack solvent-borne polyurethane coatings are prepared according the following procedure:

D amino acid mixture, in a ratio 1:1:1:1 of D-Tryosine:D-Leucine:D-Methionine:D-Tryptophan is added to the binder and solvent as mill-base formulation and stirred at high shear rate for 10 minutes until a particle size below 5 μm is achieved.

Mill-Base Formulation:

| | Weight-% |
|---|---|
| Macrynal SM 510n (60% acrylic copolymer in 10% aromatic | 88.5 |
| hydrcarbons, 20% xylene, 10% n-butylacetate) | |
| Butylglykolacetate (solvent) | 11.0 |
| D-amino acid mixture | 0.5 |
| Sum | 100.0 |

The coating formulation was prepared by mixing the ingredients of component A and adding component B at the end before application (see table below). The content of the D-amino acid mixture in total formulation is 0.1 wt. %.

| Component A: | Weight-% |
|---|---|
| Mill-base | 28.0 |
| Macrynal SM 510n (60% acrylic copolymer in 10% aromatic | 52.3 |
| hydrcarbons, 20% xylene, 10% n-butylacetate) | |
| Butylglykolacetate (solvent) | 9.7 |
| Solvesso 100 (mixture of aromatic hydrocarbons) | 6.2 |
| Methylisobutylketone (solvent) | 3.6 |
| Byk 300 (52% solution of a polyether modified | 0.2 |
| dimethylpolysiloxane-copolymer in xylene/isobutanol (4/1)) | |
| Component B: | |
| Desmodur N 75 (75% aliphatic isocyanate in | 40.0 |
| methoxypropylacetate/xylene (1/1)) | |
| Sum | 140.0 |

Each coating formulation is sprayed on white coated aluminum panels (dry film thickness: 40 μm) and dried 30 minutes at 80° C.

### Examples Related to Beauty/Personal Care Formulation

### Example 15

### Water in Oil W/O Representative Formulation

The following W/O emulsion is prepared containing 0.1% wt/wt D-amino acid mixture in a ratio 1:1:1:1 of D-Tryosine:D-Leucine:D-Methionine:D-Tryptophan.

### W/O Emulsion:

| Part A | Paraffin Liquidum | 7.5 | parts |
|---|---|---|---|
| | Isohexadecane | 6.0 | |
| | PEG-7 Hydrogenated Castor Oil | 4.1 | |
| | Isopropyl Palimitate | 2.0 | |
| | Cera microcristallina | 0.5 | |
| | Lanolin Alcohol | 0.6 | |
| Part B | Water | dil. to 100 parts | |
| | | total formulation | |
| | Magnesium Sulfate | 1.0 | |
| | Glycine | 3.20 | |
| Part C | D-amino acid mixture | 20 | parts of 0.5% |
| | | | wt/wt aqueous soln. |

### Example 16

### Oil in Water O/W Representative Formulation

The following O/W emulsion is prepared containing 0.1% wt/wt D-amino acid mixture in a ratio 1:1:1:1 of D-Tryosine:D-Leucine:D-Methionine:D-Tryptophan.

### O/W Emulsion:

| Part A | Steareth-2 | 2.2 | parts |
|---|---|---|---|
| | Steareth-21 | 1.0 | |
| | PEG-15 Stearyl Ether | 6.0 | |
| | Dicaprylyl Ether | 6.0 | |
| Part B | Water | dil. to 100 parts total | |
| | | formulation | |
| | Sodium Polyacrylate | 0.2 | |
| Part C | D-amino acid mixture | 20 | parts of 0.5% wt/wt |
| | | | aqueous soln. |

### Example 17

### In Vivo Inhibition of S. Aureus Biofilm Formation

In vivo testing of a D-amino acid or a combination of two or more D-amino acids is conducted as described in Anguita-Alonso et al., Antimicrobial Agents and Chemotherapy, 51:2594 (2007).

### Example 18

### Alternative In Vivo Inhibition of S. Aureus Biofilm Formation

In vivo testing of a D-amino acid or a combination of two or more D-amino acids is conducted as described in Beenken et al., J. Bacteriology, 186:4665 (2004).

### Example 19

### Preparation of a Stable Aqueous Mixture of D-Tyr, D-Leu, D-Typ and D-Met

Amino acids D-Met and D-Leu are dissolved individually in deionized water at room temperature using a concentration 5 mg/mL. Typically 10 mL of solution is prepared for each amino acid. D-Tryptophan is dissolved into deionized water at 5 mg/mL, but slight heating is required, 40-50° C. for 5-10 minutes. D-Tyrosine is dissolved at 5 mg/mL in 0.05M HCl and heating is required, 40-50° C. for 5-10 minutes. A heated sonication bath can be used to aid in the solution of the amino acids. All solutions are combined and sterile filtered at room temperature resulting in about 40 mL of stock solution.

### Example 20

### Preparation of a Stable Aqueous Mixture of D-Tyr, D-Pro, and D-Phe

An aqueous solution is prepared as described in Example 19.

### Example 21

### Preparation of a Stable Aqueous Mixture of D-Tyr, D-Asp, and D-Glu

An aqueous solution is prepared as described in Example 19.

### Example 22

### Preparation of a Stable Aqueous Mixture of D-Tyr, D-Arg, D-His, and D-Lys

An aqueous solution is prepared as described in Example 19.

### Example 23

### Preparation of a Stable Aqueous Mixture of D-Tyr, D-Ile, D-Val- and D-Asn

An aqueous solution is prepared as described in Example 19.

### Example 24

### Preparation of a Stable Aqueous Mixture of D-Tyr, D-Cys, D-Ser, D-Thr and D-Gln

An aqueous solution is prepared as described in Example 19.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a biofilm-related disorder in a subject in need thereof, the method comprising administering to the subject a composition comprising an effective amount of a D-amino acid, or a pharmaceutically acceptable salt, ester, or derivative thereof, said composition being essentially free of the corresponding L-amino acid, thereby treating the biofilm-related disorder,
wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, D-asparagine and a combination thereof.

2. A method of treating a biofilm-related disorder in a subject in need thereof, the method comprising administering to the subject a composition comprising an effective amount of a combination of two or more D-amino acids, or pharmaceutically acceptable salts, esters, or derivatives thereof, thereby treating the biofilm-related disorder.

3. The method of claim 2,
wherein the combination of D-amino acids is a combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-asparagine, and D-tyrosine.

4. The method of any of claim 1, 2 or 3, wherein the composition is administered to a surface of the subject selected from the group of dermal and mucosal surfaces and combinations thereof.

5. The method of claim 4, wherein the surface is an oral surface, a skin surface, a urinary tract surface, a vaginal tract surface, or a lung surface.

6. The method of claim 1, 2 or 3, wherein the composition is administered to the subject via subcutaneous, intra-muscular, intra-peritoneal, intravenous, oral, nasal, or topical administration, and a combination thereof.

7. The method any of claims 1-6, wherein the subject is a human.

8. The method of any of claims 1-7, wherein the formation of a biofilm is inhibited.

9. The method of any of claims 1-7, wherein a previously formed biofilm is disrupted.

10. The method of any of claims 1-9, wherein the D-amino acid is administered at a concentration of 0.1 nM to 100 μM.

11. The method of any of claim 1, 2 or 3, wherein the biofilm-related disorder is selected from the group consisting of pneumonia, cystic fibrosis, otitis media, chronic obstructive pulmonary disease, and a urinary tract infection and combinations thereof.

12. The method of claim 1, 2 or 3, wherein the biofilm-related disorder is a medical device-related infection.

13. The method of any of claims 1-12, wherein the biofilm-related disorder is caused by bacteria.

14. A method of treating, reducing, or inhibiting biofilm formation by bacteria on a biologically-related surface, the method comprising:
contacting a biological surface with a composition comprising an effective amount of a D-amino acid, or a pharmaceutically acceptable salt, ester, or derivative thereof, said composition being essentially free of the corresponding L-amino acid, thereby treating, reducing or inhibiting formation of the biofilm,
wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, D-asparagine and a combination thereof.

15. A method of treating, reducing, or inhibiting biofilm formation by bacteria on a biologically-related surface, the method comprising:
contacting a biological surface with a composition comprising an effective amount of a combination of two or more D-amino acids, or a pharmaceutically acceptable salts, esters, or derivatives thereof, reducing or inhibiting formation of the biofilm.

16. The method of claim 15,
wherein the combination of D-amino acids is a combination of two or more D-amino acids selected from the group consisting D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-asparagine, and D-tyrosine.

17. The method of claim 14 or 15, wherein the surface comprises a medical device, a wound dressing, a contact lens, or an oral device.

18. The method of claim 17, wherein the medical device is selected from the group consisting of a clamp, forcep, scissors, skin hook, tubing, needle, retractor, scaler, drill, chisel, rasp, saw, catheter, orthopedic device, artificial heart valve, prosthetic joint, voice prosthetic, stent, shunt, pacemaker, surgical pin, respirator, ventilator, and an endoscope and combinations thereof.

19. The method of any one of the preceding claims, wherein the bacteria are Gram-negative or Gram-positive bacteria.

20. The method of claim 19, wherein the bacteria are of the genus Actinobacillus, Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Helicobacter, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia, or Xanthomonas.

21. The method of any one of the preceding claims, wherein the composition comprises D-tyrosine.

22. The method of claim 21, wherein the composition further comprises one or more of D-proline and D-phenylalanine.

23. The method of claim 21, wherein the composition further comprises one or more of D-leucine, D-tryptophan, and D-methionine.

24. The method of claim 21, wherein the composition further comprises one or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-asparagine.

25. The method of claim 21, wherein the composition comprises D-tyrosine, D-proline and D-phenylanalilne.

26. The method of claim 21, wherein the composition comprises D-tyrosine, D-leucine, D-trytophan and D-methionine.

27. The method of any one of the preceding claims, further comprising administering a biocide.

28. The method of claim 27, wherein the biocide is an antibiotic.

29. The method of any one of the preceding claims, wherein the composition is essentially free of detergent.

30. A composition comprising:
a D-amino acid in an amount effective to treat, reduce, or inhibit biofilm formation, said composition being essentially free of the corresponding L-amino acid,
wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, D-asparagine and a combination thereof.

31. A composition comprising:
a combination of two or more D-amino acids in an amount effective to treat, reduce, or inhibit biofilm formation.

32. The composition of claim 31,
wherein the combination of D-amino acids is a combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-asparagine, and D-tyrosine.

33. The composition of claim 30, 31 or 32, wherein the D-amino acid is D-tyrosine.

34. The composition of claim 33, wherein the composition further comprises one or more of D-proline and D-phenylalanine.

35. The composition of claim 33, wherein the composition further comprises one or more of D-leucine, D-tryptophan, and D-methionine.

36. The composition of claim 33, wherein the composition further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-tyrosine.utamic acid, D-phenylalanine, D-histidine, D-isoleucine, D-lysine, D-leucine, D-asparagine, D-proline, D-glutamine, D-arginine, D-serine, D-threonine, D-valine, and D-tryptophan.

37. The composition of claim 33, wherein the composition comprises D-tyrosine, D-proline and D-phenylanalilne.

38. The composition of claim 33, wherein the composition comprises D-tyrosine, D-leucine, D-trytophan and D-methionine.

39. The composition of any one of claims 30-38, wherein the composition comprises polyhexamethylene biguanide, chlorhexidine, xylitol, triclosan, or chlorine dioxide.

40. The composition of any one of claims 30-38, further comprising a pharmaceutically acceptable carrier.

41. The composition of any one of claims 30-40, wherein the effective amount is an amount effective to treat or prevent a biofilm-related disorder.

42. The composition of claim 41, wherein the biofilm-related disorder is pneumonia, cystic fibrosis, otitis media, chronic obstructive pulmonary disease, or a urinary tract infection.

43. The composition of claim 41, wherein the biofilm-related disorder is a medical device-related infection.

44. The composition of claim of any one of claims 30-40, wherein an effective amount comprises and amount effective to treat or prevent a biofilm on a surface.

45. The composition of claim 44, wherein the composition further comprises an agent suitable for application to the surface.

46. The composition of any of claims 30-45, wherein the composition is formulated as a wash solution, a dressing, a wound gel, or a synthetic tissue.

47. The composition of any of claims 30 to 45, wherein the composition is formulated as tablets, pills, troches, capsules, aerosol spray, solutions, suspensions, gels, pastes, creams, or foams.

48. The composition of any of claims 30 to 45, wherein the composition is formulated for parenteral, e.g., intravenous, intradermal, subcutaneous, oral (e.g., inhalation), transdermal (topical), transmucosal, vaginal and rectal administration.

49. A biofilm resistant medical device, comprising:
a surface likely to contact a biological fluid; and
a D-amino acid coated on or impregnated into said surface, wherein the D-amino acid is in an amount effective to treat, reduce, or inhibit biofilm formation, said coating being essentially free of the corresponding L-amino acid,
wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, D-asparagine and a combination thereof.

50. A biofilm resistant medical device, comprising:
a surface likely to contact a biological fluid; and
a combination of D-amino acids coated on or impregnated into said surface, wherein the combination of D-amino acids is in an amount effective to treat, reduce, or inhibit biofilm formation.

51. The device of claim 50,
wherein the combination of D-amino acids is a combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-asparagine, and D-tyrosine.

52. The device of any one of claim 49, 50 or 51, wherein the coating comprises D-tyrosine.

53. The device of claim 52, wherein the coating further comprises one or more of D-proline and D-phenylalanine.

54. The device of claim 52, wherein the coating further comprises one or more of D-leucine, D-tryptophan, and D-methionine.

55. The device of claim 52, wherein the coating further comprises one or more of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-asparagine.

56. The device of any of claims 49 through 55, wherein the D-amino acid is formulated as a slow-release formulation.

57. The device of any of claims 49 through 56, wherein the surface is essentially free of detergent.

58. The device of any of claims 49 through 57, wherein the device is selected from one or more of clamp, forcep, scissors, skin hook, tubing, needle, retractor, scaler, drill, chisel, rasp, saw, catheter, orthopedic device, artificial heart valve, prosthetic joint, voice prosthetic, stent, shunt, pacemaker, surgical pin, respirator, ventilator and endoscope.

59. A potable liquid comprising a D-amino acid at a concentration in the range of 0.000001% to 0.5%,
wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-tyrosine, D-asparagine and a combination thereof.

60. A potable liquid comprising a combination of D-amino acids at a concentration in the range of 0.000001% to 0.5%,
wherein the combination of D-amino acids is a combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, D-asparagine, and D-tyrosine.

61. A composition resistant to biofilm formation, comprising:
a pharmaceutically or cosmetically suitable base; and
an effective amount of a D-amino acid distributed in the base, thereby treating, reducing or inhibiting formation of the biofilm,
wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-asparagine, and D-tyrosine, and a combination thereof,
wherein the base is essentially free of the corresponding L-amino acid.

62. A composition resistant to biofilm formation, comprising:
a pharmaceutically or cosmetically suitable base; and
an effective amount of a combination of D-amino acids distributed in the base, thereby treating, reducing or inhibiting formation of the biofilm,
wherein the combination of D-amino acids is a combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

63. The composition of claim 61 or 62, wherein the base is selected from a liquid, gel, paste, or powder.

64. The composition of claim 63, wherein the composition is selected from the group consisting of shampoos, bath additives, hair care preparations, soaps, lotions, creams, deodorants, skin-care preparations, cosmetic personal care preparations, intimate hygiene preparations, foot care preparations, light protective preparations, skin tanning preparations, insect repellants, antiperspirants, sharing preparations, hair removal preparations, fragrance preparations, dental care, denture care and mouth care preparations and combinations thereof.

65. An oral composition comprising:
an orally acceptable carrier; and
an effective amount of a D-amino acid, thereby treating, reducing or inhibiting formation of the biofilm,
wherein the D-amino acid is selected from the group consisting of D-alanine, D-cysteine, D-aspartic acid, D-glutamic acid, D-histidine, D-isoleucine, D-lysine, D-leucine, D-asparagine, D-proline, D-glutamine, D-arginine, D-serine, D-threonine, D-valine, D-tryptophan, D-asparagine, and D-tyrosine, and a combination thereof,
wherein the composition is essentially free of the corresponding L-amino acid.

66. An oral composition comprising:
an orally acceptable carrier; and
an effective amount of a combination of D-amino acids, thereby treating, reducing or inhibiting formation of the biofilm,
wherein the combination of D-amino acids is a combination of two or more D-amino acids selected from the group consisting of D-alanine, D-cysteine, D-aspartic acid, D-glutamic acid, D-phenylalanine, D-histidine, D-isoleucine, D-lysine, D-leucine, D-methionine, D-asparagine, D-proline, D-glutamine, D-arginine, D-serine, D-threonine, D-valine, D-tryptophan, and D-tyrosine.

67. The oral composition of claim 65 or 66, wherein the oral composition is in the form of a toothpaste, tooth gel, or tooth powder.

68. The oral composition of claim 65 or 66, wherein the oral composition is in the form of a mouthwash, mouth rinse, mouth spray, a dental solution, or an irrigation fluid.
